(19) United States
(12) Patent Application Publication
(10) Pub. No.: US 2022/0307080 A1
(43) Pub. Date: Sep. 29, 2022

(54) METHODS AND REAGENTS FOR NUCLEIC ACID AMPLIFICATION AND/OR DETECTION

(71) Applicant: SIMON FRASER UNIVERSITY, Burnaby (CA)

(72) Inventors: Peter J. UNRAU, Coquitlam (CA); Amir ABDOLAHZADEH, Coquitlam (CA)

(21) Appl. No.: 17/616,841

(22) PCT Filed: Jun. 7, 2020
(86) PCT No.: PCT/IB2020/055348

§ 371 Date: Dec. 6, 2021

Related U.S. Application Data
(60) Provisional application No. 62/858,874, filed on Jun. 7, 2019.

(51) Int. Cl. C12Q 1/6853; C12Q 1/70; C12Q 1/689; C12N 15/11

(52) U.S. Cl. CPC C12Q 1/6853; C12Q 1/70; C12Q 1/689; C12N 15/111; C12N 2310/16; C12Q 2600/106

(57) ABSTRACT

The present invention relates to the amplification and/or detection of nucleic acid molecules. More specifically, the present invention relates to the sensitive amplification, detection, and/or quantification of nucleic acid molecules.

### FIELD

The present invention relates to the amplification and/or detection of nucleic acid molecules. More specifically, the present invention relates to the sensitive amplification, detection, and/or quantification of nucleic acid molecules.

### BACKGROUND

Infectious diseases caused by pathogenic microorganisms, such as bacteria, viruses and eukaryotic parasites, are among the most serious public health concerns worldwide. Successful methods for disease diagnosis and treatment, food safety control and environmental monitoring, therefore, require rapid and specific identification of the infectious agent. Simplicity and low cost are equally important. Methods that do not rely on high-end instrumentation and skilled personnel, for example, can be employed in settings where COVID19, HIV, TB, malaria, outbreaks of certain types of influenza A and Ebola viruses pose great risk to patient care, settings, where advanced diagnostic technologies are limited or nonexistent due to economical constraints^{1,2}.

Traditional methods for pathogen detection involve culturing of microorganisms on agar plates followed by standard biochemical identifications, which, although inexpensive and simple, are laborious and time consuming³. They often require 2 to 3 days of preliminary identification and more than a week for the pathogen identity confirmation, which slows down effective diagnosis^{4,5}. This delay has a major impact on morbidity and mortality rates. Misdiagnosed therapies have been shown to reduce survival for serious infections five-fold⁶. Moreover, these methods can be limited by their low sensitivity^{7,8}. Also, culture methods can only identify organisms that are capable of growing in culture and cannot detect viable but culture-negative pathogens^{6,4}. These, however, can be identified using molecular nucleic acid detection methods.

A variety of applications involving pathogen detection have extensively used nucleic acids as biomarkers^{5,1,4}. Due to their multipurpose functions and broad applications, a number of methods have been developed to detect extremely small amounts of nucleic acids in complex biological samples⁴. Between RNA and DNA, RNA detection is of particular interest as many living pathogens carry multiple copies of RNA (in the case of ribosomal RNA, thousands) which gives greater initial template concentration for amplification, as well as being the only source of genetic information in some high profile viral pathogens (Measles virus, Influenza, and HIV to name a few). Nucleic acid testing (NAT) is rapid and intrinsically more specific and sensitive over conventional methods. In addition, it can be used to identify microorganisms directly in clinical specimens without culturing, significantly shortening detection times. Rapid pathogen detection translates into shorter hospital stay, improved patient treatment, prevention of community outbreaks and epidemics of global nature.

The goal of NAT is to identify and potentially quantify specific nucleic acid sequences from clinical samples. This technology has traditionally involved three steps—nucleic acid isolation, amplification and detection. However, with the advent of fluorescent DNA probes and intercalating dyes that allow real time quantification of amplification products, amplification and detection can now be combined in one step, considerably shortening detection times.

Polymerase chain reaction (PCR) was the first and remains the most popular amplification technology for amplifying and detecting low abundance nucleic acids. Invented nearly 30 years ago⁹, it is capable of detecting specific target DNA sequences corresponding to single bacterial pathogens^{10,11}. The PCR amplification products can be visualized with electrophoresis gel stained with intercalating fluorescent dyes. PCR variations include multiplex PCR (mPCR) and real-time or quantitative PCR (qPCR). Multiplex PCR offers a more rapid detection as compared to simple PCR, since it simultaneously amplifies multiple targets with several set of primers. Primer design and concentration are of particular importance in avoiding primer dimerization and producing reliable PCR product. In comparison, qPCR does not require gel electrophoresis for the detection, but monitors product formation continuously by measuring fluorescence produced by intercalating dyes (such as SYBR Green) dual labelled probes (Taqman) or molecular beacons¹². For pathogens with RNA genomes, RT-PCR is employed, which uses RNA as a template for the production of cDNA, which is, in turn, amplified by PCR¹³. Albeit highly sensitive and specific, various PCR methods are affected by PCR inhibitors present in the nucleic acid prep, are costly due to the need for thermocycling equipment and fluorescent probes, and are time consuming⁴.

Isothermal amplification of nucleic acids (INA) is an alternative to PCR, where amplification is achieved at a constant temperature without the need for thermocycling, making it both less complex and less expensive^{14,15}. INA methods can be performed in a broad range of conditions, such as a water bath or equivalent fixed temperature heating device, can be performed inside the cell or on a cell surface, PCR¹⁴. INA reactions can be classified based on their reaction kinetics as exponential (e.g., Nucleic acid sequence based amplification (NASBA)¹⁶, Rolling Circle Amplification (RCA)¹⁴, Loop mediated isothermal amplification (LAMP)¹⁷, Recombinase polymerase amplification (RPA)¹⁸, Helicase dependent amplification (HDA)¹⁹), Nicking Enzyme Amplification (NEAR)³⁵, Strand Displacement Amplification (SDA)³⁶, or linear and cascade amplification methods. As with qPCR, INA reactions can be analyzed while the reaction is progressing, which can shorten the reaction time, albeit making it more complex in terms of instrumentation.

NASBA¹⁶ utilizes three enzymes to amplify an RNA product isothermally at 41° C. First, a primer containing a T7 promoter hybridizes to a target RNA and is extended by a reverse transcriptase (RT). RNase H then degrades the hybridized RNA to leave the bare cDNA. Next, a second primer hybridizes to the cDNA and is extended by the RT to the end of the initial hybridizing primer, producing a dsDNA containing a T7 promoter. T7 RNA polymerase then transcribes an RNA encoded between the regions where the primers originally annealed used. As multiple copies of RNA are made, free primer can continue to hybridize, be extended, and produce more template. This results in exponential amplification of the DNA template and RNA product.

Rolling circle amplification (RCA)¹⁴ involves a DNA or RNA polymerase that uses a circular DNA template to generate long RNA/DNA products. The circular template typically contains a polymerase promoter, hybridization sites, and template for a product that can act as a reporter (commonly a target site for a hybridization-based reporter, such as a molecular beacon). Unlike transcription with a linear target which produces a single copy of product, the polymerase can complete full circles of the circular template producing many copies. A method for exponential amplification involves hybridization oligonucleotides hybridizing to a target sequence, their ligation to form a closed circular template, and multiple copy production by a polymerase, the newly generated product containing multiple copies of the target sequence, which can act as new templates for linear template hybridization.

Loop mediated isothermal amplification (LAMP)¹⁷ utilizes two or three sets of primers with a strand displacing DNA polymerase to isothermally produce multiple mixed species of DNA product isothermally at 60-65° C. This method relies on producing DNA products containing single-stranded loop regions that allow for hybridization of primers to an already extended DNA product. The addition of a reverse transcriptase allows for detection of RNA samples.

Recombinase polymerase amplification (RPA)¹⁸ relies on three enzymes and is able to amplify a DNA product isothermally at 37° C., producing many DNA copies. Initially, recombinase proteins guide a primer strand to hybridize to a DNA template. Single stranded binding proteins (SSB) bind to the strand of the DNA duplex being displaced and further the displacement. Next, a DNA polymerase extends the primer forming a new duplex. The same reaction occurs on the opposite strand, thus, leading to a complete duplication of the DNA molecule. These steps cyclically continue for exponential amplification. RPA has been multiplexed with LAMP for the detection of multiple targets simultaneously²⁰.

Helicase dependent amplification (HDA)¹⁹ is an isothermal amplification method that requires the use of a DNA helicase. Essentially, this system functions similarly to PCR, in that it is dependent on melting of strands, annealing of primers, and extension by a polymerase. Whereas PCR requires changes in temperature to aid the process of amplification, HDA relies on enzymatic processing. First, DNA helicase melts two stranded DNA complexes. Second, primers are allowed to hybridize to the target DNA. Third, a strand displacing DNA polymerase extends the primers to complete a new DNA duplex. This process repeats for exponential amplification at 37° C.

Nicking Enzyme Amplification (NEAR)³⁵ and Strand Displacement Amplification (SDA)³⁶ are isothermal methods that amplify DNA at constant temperature (55° C. to 59° C.) using strand displacing DNA polymerase (Bst DNA polymerase, Large Fragment or Klenow Fragment (3′-5′ exo-) and a nicking enzyme. Nicks are created by strand-limited restriction endonuclease at a site contained within a primer. The nick is generated with each polymerase displacement step, resulting in exponential amplification.

Amplifying very low concentrations of nucleic acid is a challenging problem and it has been known for some time that it is difficult to isothermally amplify RNA templates in the absence of accompanying amplification artifacts¹⁵. One attempt at addressing this issue uses the SHERLOCK approach, where the products of RPA based amplification are screened for the desired amplicon using a CRISPR-mediated cleavage mechanism to specifically cleave a fluorescently tagged reporter construct^{21,22}. While increasing sensitivity and enabling SNP based specificity, the additional enzymatic step and the requirement for a fluorescent reporter adds significant complexity to the detection of RNA.

SHERLOCK and DETECTR^{23,21} utilize an initial isothermal amplification system (RPA) to amplify a target using a primer set that includes a T7 promoter and guide RNA cassette sequences. The product of the RPA is transcribed using T7 RNA polymerase leading to the production of multiple copies of guide RNA. The guide RNA then guides Cas13a proteins to detect RNA species, resulting in activation of the Cas13a for the non-specific degradation of RNA species, in this case degrading RNA molecular beacons and releasing a fluorescent signal (SHERLOCK). Alternatively, the guide RNA can guide Cas12a to target an RNA molecule, activating the enzyme for non-specific cleavage of DNA molecular beacons, also resulting in fluorescence (DETECTR). These technologies can be adapted to detect RNA species by the addition of a reverse transcriptase in the initial RPA reaction. In total, these systems require five enzymes and a reporter for detection of a DNA and an additional enzyme for the detection of RNA species.

RNA tags, such as fluorogenic RNA aptamers, can be used to label RNAs of interest. RNA aptamers for fluorogenic compounds that generate fluorescence upon binding can be selected using in vitro selection to optimize both the fluorescent enhancement of the fluorogenic aptamer system (F_{E}) and the K_{D} of the aptamer-fluorophore interaction^{24,25}. Maximizing both parameters gives fluorogenic aptamers higher intrinsic contrast than the MS2-fluorescent protein recruiting type systems^{26,27,28.} As fluorophore ligands are inexpensive and since the RNA fluorogenic aptamer can be made by transcription, fluorogenic aptamers potentially offer many intrinsic advantages as reporters.

The RNA Mango aptamer series have extremely high contrast making them useful in vitro fluorescent reporters. These aptamers have nanomolar binding affinity to a thiazole orange-based ligand (TO1-Biotin) that is capable of becoming up to 4,000 times brighter upon binding an RNA Mango aptamer^{29,30,31}. Of particular note, the second generation of RNA Mango aptamers (Mango II, III, and IV) are highly resistant to the magnesium ion concentrations, which is typically found in in vitro assays and, also, work in a range of monovalent metal ion concentrations³⁰. Mango III has also been recently improved by structure guided engineering to become even brighter³².

### SUMMARY

The present invention relates to the amplification and/or detection of nucleic acid molecules.

In one aspect, the present invention provides a nucleic acid molecule, or analog thereof, including: a first nucleic acid sequence, capable of hybridizing to at least a portion of a target nucleic acid sequence, or reverse-complement thereof, and further including an aptamer-encoding template sequence, where the aptamer-encoding template sequence is positioned at the 3′ end of the first nucleic acid sequence; and a second nucleic acid sequence, capable of hybridizing to at least a portion of a target nucleic acid sequence, or reverse-complement thereof, wherein the 5′ end of the second nucleic acid sequence is covalently attached to the 3′ end of the first nucleic acid sequence, and where the 3′ end of the second nucleic acid sequence does not substantially hybridize to the first nucleic acid sequence.

In some embodiments, at least the terminal three nucleotides of the 3′ end of the second nucleic acid sequence do not hybridize to the first nucleic acid sequence.

In some embodiments, the first nucleic acid sequence may be about 20 to about 100 nucleotides in length.

In some embodiments, the aptamer-encoding template sequence may encode a fluorogenic aptamer sequence.

In some embodiments, the fluorogenic aptamer sequence may have a fluorophore binding dissociation constant (K_{D}) of about 0.01 nM to about 100 nM.

In some embodiments, the nucleic acid molecule may include a terminal stem structure, where at least the terminal nucleotide of the 5′ end of the second nucleic acid sequence may be complementary to at least the terminal nucleotide of the 5′ end of the first nucleic acid to form at least a portion of the terminal stem structure.

In some embodiments, at least the terminal two or three nucleotides of the 5′ end of the second nucleic acid sequence may be complementary to at least the terminal two or three nucleotides of the 5′ end of the first nucleic acid to form at least a portion of the terminal stem structure.

In some embodiments, the nucleic acid molecule, or analog thereof, may be DNA-based or RNA-based.

In some embodiments, the second nucleic acid sequence may include a degenerate sequence.

In some embodiments, the nucleic acid molecule does not include an RNA polymerase promoter sequence.

In some embodiments, the target nucleic acid sequence may be from a virus, a microorganism, a fungus, an animal or a plant, or may be a synthetic construct.

In some embodiments, the target nucleic acid sequence may be from a pathogenic virus or a pathogenic bacterium.

In another aspect, the present invention provides a composition including a first nucleic acid molecule as described herein.

In some embodiments, the composition may further include a second nucleic acid molecule capable of hybridizing to at least a portion of a target nucleic acid sequence, or reverse-complement thereof, and including a first RNA polymerase promoter sequence, where the first and second nucleic acid molecules form a first primer pair capable of amplifying a first sequence of the target nucleic acid sequence.

In some embodiments, the 3′ end of the first nucleic acid molecule may not substantially hybridize to the second nucleic acid molecule or to itself.

In some embodiments, the first and second nucleic acid molecules may not substantially hybridize to each other.

In some embodiments, the terminal one, two or three bases of the 3′ end of the first nucleic acid molecule may hybridize to the terminal one, two or three bases of the 3′ end of the second nucleic acid molecule.

In some embodiments, the 3′ end of the first nucleic acid molecule may be contiguous with the 3′ end of the second nucleic acid molecule when aligned with the sequence of the target nucleic acid.

In some embodiments, the composition as described herein may further include a third nucleic acid molecule and a fourth nucleic acid molecule, where the third and fourth nucleic acid molecules form a second primer pair capable of amplifying a second sequence of the target nucleic acid molecule, where either the third nucleic acid molecule or the fourth nucleic acid molecule may include a second RNA polymerase promoter sequence, and where the second primer pair may hybridize to the target nucleic acid molecule at locations external to that of the first primer pair and may be capable of amplifying the first sequence and the second sequence.

In some embodiments, the second RNA polymerase promoter sequence may transcribe the second sequence of the target nucleic acid molecule in a direction opposite to that of the second nucleic acid molecule.

In some embodiments, when the third nucleic acid molecule includes the second RNA polymerase promoter sequence, the fourth nucleic acid molecule includes a second aptamer-encoding sequence, or when the fourth nucleic acid molecule includes the second RNA polymerase promoter sequence, the third nucleic acid molecule includes a second aptamer-encoding sequence.

In some embodiments, the 3′ end of the third nucleic acid molecule may not substantially hybridize to the fourth nucleic acid molecule.

In some embodiments, the third and fourth nucleic acid molecules may not substantially hybridize to each other.

In some embodiments, the 3′ ends of the first, second, third and fourth nucleic acid molecules may not substantially hybridize to each other.

In some embodiments, the first, second, third and fourth nucleic acid molecules may not substantially hybridize to each other.

In some embodiments, the composition as described herein may further include a fifth nucleic acid molecule and a sixth nucleic acid molecule, where the fifth and sixth nucleic acid molecules may form a third primer pair capable of amplifying a third sequence of the target nucleic acid molecule, where either the fifth nucleic acid molecule or the sixth nucleic acid molecule may include a third RNA polymerase promoter sequence, where the third primer pair may hybridize to the target nucleic acid molecule at a location external to that of the first and second primer pairs and may be capable of amplifying the first, second and third sequences.

In some embodiments, the third RNA polymerase promoter sequence may transcribe the third sequence of the target nucleic acid molecule in the same direction as the second nucleic acid molecule.

In some embodiments, when the fifth nucleic acid molecule includes the third RNA polymerase promoter sequence, the fourth nucleic acid molecule includes a third aptamer-encoding sequence, or when the fourth nucleic acid molecule includes the third RNA polymerase promoter sequence, the fifth nucleic acid molecule includes a third aptamer-encoding sequence.

In some embodiments, the 3′ end of the fifth nucleic acid molecule may not substantially hybridize to the 3′ end of the fourth nucleic acid molecule.

In some embodiments, the fifth and fourth nucleic acid molecules may not substantially hybridize to each other.

In some embodiments, the 3′ ends of the first, second, third, fourth, fifth and sixth nucleic acid molecules may not substantially hybridize to each other.

In some embodiments, the first, second, third, fourth, fifth and sixth nucleic acid molecules may not substantially hybridize to each other.

In some embodiments, the composition as described herein may include one or more nucleic acid molecules comprising a sequence as set forth in Table 3.

In some embodiments, one or more of the nucleic acid molecules may be premixed.

In some embodiments, one or more of the nucleic acid molecules may be provided in a liquid.

In some embodiments, one or more of the nucleic acid molecules may be lyophilized.

In another aspect, the present invention provides a kit include one or more of the nucleic acid molecules or compositions, as described herein, together with instructions for amplification of a target nucleic acid sequence.

In some embodiments, the amplification may be an isothermal amplification, such as nucleic acid sequence based amplification, Rolling Circle Amplification, Loop mediated isothermal amplification, Helicase dependent amplification, or Strand Displacement Amplification.

In another aspect, the present invention provides a method of amplifying a target nucleic acid sequence, the method including: providing a sample suspected of containing a target nucleic acid molecule; providing a first nucleic acid molecule as described herein; providing a second nucleic acid molecule capable of hybridizing to at least a portion of the target nucleic acid sequence, or complement thereof, and including a first RNA polymerase promoter sequence, where the first and second nucleic acid molecules form a first primer pair capable of amplifying a first sequence of the target nucleic acid sequence; and performing a first amplification reaction including the target nucleic acid molecule and the first primer pair to obtain a first amplification product, where the first amplification product includes the first sequence of the target nucleic acid sequence.

In some embodiments, the 3′ end of the first nucleic acid molecule may not substantially hybridize to the 3′ end of the second nucleic acid molecule.

In some embodiments, the first and second nucleic acid molecules may not substantially hybridize to each other.

In some embodiments, the terminal one, two or three bases of the 3′ end of the first nucleic acid molecule may hybridize to the terminal one, two or three bases of the 3′ end of the second nucleic acid molecule.

In some embodiments, the 3′ end of the first nucleic acid molecule may be contiguous with the 3′ end of the second nucleic acid molecule when aligned with the sequence of the target nucleic acid.

In some embodiments, the method may further include: providing a third nucleic acid molecule and a fourth nucleic acid molecule, where the third and fourth nucleic acid molecules form a second primer pair capable of amplifying a second sequence of the target nucleic acid molecule, where either the third nucleic acid molecule or the fourth nucleic acid molecule includes a second RNA polymerase promoter sequence, where the second primer pair may hybridize to the target nucleic acid molecule at a location external to that of the first primer pair and may be capable of amplifying the first sequence and the second sequence of the target nucleic acid molecule; and performing a second amplification reaction including the first amplification product and the second primer pair to obtain a second amplification product, where the second amplification reaction may be performed prior to the first amplification reaction and where the second amplification product may include the first sequence and the second sequence of the target nucleic acid molecule.

In some embodiments, the second RNA polymerase promoter sequence may transcribe the second sequence of the target nucleic acid molecule in a direction opposite to that of the second nucleic acid molecule.

In some embodiments, when the third nucleic acid molecule includes the second RNA polymerase promoter sequence, the fourth nucleic acid molecule includes a second aptamer-encoding sequence, or when the fourth nucleic acid molecule includes the second RNA polymerase promoter sequence, the third nucleic acid molecule includes a second aptamer-encoding sequence.

In some embodiments, the 3′ end of the third nucleic acid molecule may not substantially hybridize to the 3′ end of the fourth nucleic acid molecule.

In some embodiments, the third and fourth nucleic acid molecules may not substantially hybridize to each other.

In some embodiments, the 3′ ends of the first, second, third and fourth nucleic acid molecules may not substantially hybridize to each other.

In some embodiments, the first, second, third and fourth nucleic acid molecules may not substantially hybridize to each other.

In some embodiments, the method as described herein further includes detecting the target nucleic acid sequence.

In some embodiments, method as described herein further includes quantifying the target nucleic acid sequence.

In some embodiments, the amplification may be an isothermal amplification, such as nucleic acid sequence-based amplification, Rolling Circle Amplification, Loop mediated isothermal amplification, Helicase dependent amplification, Strand Displacement Amplification, or combination thereof.

In some embodiments, the amplification may be RNA based or DNA based.

In some embodiments, the amplification may be multiplexed.

In some embodiments, the amplification may include at least two colour imaging.

In some embodiments, the amplification may include at least three colour imaging.

In some embodiments, the sample may be from a virus, a microorganism, a fungus, an animal, a plant or from the environment.

In some embodiments, the sample may be from a pathogenic virus, such as a coronavirus (e.g., SARS, MERS or SARS-CoV-2) or a pathogenic bacterium.

In some embodiments, the sample may be obtained from water, soil, saliva, feces, urine, blood, tracheal aspirate or nasal aspirate.

In some embodiments, the animal may be a human.

In another aspect, the present invention provides a method of detecting a target nucleic acid molecule, by providing a sample including a nucleic acid molecule; and amplifying the nucleic acid molecule by isothermal nucleic acid amplification (INA), where the amplifying includes the use of nested oligonucleotide primer pairs.

In some embodiments, the nested oligonucleotide primer pairs may include fluorogenic aptamer sequences. In some embodiments, the detection may be highly sensitive.

In alternative aspects, the present invention provides a kit including nested oligonucleotide primer pairs, where the nested oligonucleotide primer pairs may include fluorogenic aptamer sequences, together with instructions for use in an isothermal nucleic acid amplification method.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows insertion of RNA fluorogenic aptamers into RNA producing isothermal amplification systems. A. Traditional NASBA uses two primers to produce an RNA product. Artifacts are also commonly produced (black and grey products). B. fluorogenic aptamer-NASBA system features the addition of fluorogenic aptamer template sequence on the top strand (PB) primer resulting in the production of an RNA product containing a fluorescent fluorogenic aptamer tag after T7 transcription. C. Nested fluorogenic aptamer-NASBA features an outer primer NASBA reaction whose products are then diluted and fed into an inner fluorogenic aptamer-NASBA reaction (shown here using but not limited to Mango aptamers).

FIG. 2 shows nested-fluorogenic aptamer NASBA is sensitive and specific to target RNA sequence, and is robust even when an unrelated nucleic acid background is added. A. Un-nested outer (E. coli CIpB RNA) and B. inner un-nested fluorogenic aptamer NASBA (P. Fluorescens CIpB RNA) reactions. C. Nested RNA fluorogenic aptamer NASBA dramatically improves sensitivity. E. coli primers with E. coli target (Ec/Ec left set of gray bars). Using the same E. coli primers, P. fluorescence target was added (Ec/Pf middle set of dark gray bars) instead of E. coli target. P. fluorescens primers with P. fluorescens target (Pf/Pf right set of lightest gray bars). D. Nested-fluorogenic aptamer NASBA using E. coli primers was performed with inner NASBA time course shown. Black—No template added, top most light grey—150 E. coli target molecules/μL reaction, Lower light grey—5 ng/μL of A549 Human Lung Carcinoma total nucleic acid, Grey—150 E. coli target molecules/μL in the presence of 5 ng/μL of A549 Human Lung Carcinoma total nucleic acid. Error bars reflect the standard deviation of three replicates for all panels.

FIG. 3 shows a schematic showing how nesting NASBA primers increases specificity. A: Nested PCR (left) results in higher specificity due to the requirement of a second set of inner primers that hybridize within the first pair of outer primers. A nested isothermal reaction (right) can improve specificity to a target. B: Schematic of the process of nested fluorogenic aptamer NASBA.

FIG. 4 shows un-nested Outer fluorogenic aptamer NASBA fluorescence emergence is relatively insensitive. A. schematic of outer fluorogenic aptamer NASBA; B. Un-nested E. coli Outer fluorogenic aptamer NASBA primers with an E. coli target (Ec O/Ec); Values at dotted line (40 min time point) for Ec O/Ec were taken and plotted as FIG. 2A.

FIG. 5 shows un-nested inner fluorogenic aptamer NASBA fluorescence emergence with P. fluorescens is also relatively insensitive. A. schematic of inner fluorogenic aptamer NASBA. B. P. fluorescens Inner fluorogenic aptamer NASBA primers with a P. fluorescens inner target (Pf/Pf/Inner). Values at dotted line (40 min time point) for Ec O/Ec were taken and plotted as FIG. 2B.

FIG. 6 shows nested fluorogenic aptamer NASBA fluorescence is sensitive and specific. E. coli primers with E. coli CIpB RNA target (Ec/Ec table heading). Using the same E. coli primers, P. fluorescence target was added (Ec/Pf table heading) instead of E. coli target. P. fluorescens primers with P. fluorescens target (Pf/Pf table heading). All traces show the time dependence of the relevant inner fluorogenic aptamer NASBA reaction. Template concentrations are specified in RNA molecules/μl of the relevant outer reaction. Values at indicated dotted lines (100 min time point) were taken and plotted as FIG. 2C.

FIG. 7 shows E. coli RNA detection in MCF7 human tissue culture media using nested fluorogenic aptamer NASBA. Nested fluorogenic aptamer NASBA of a dilution series of E. coli cell extract from human tissue culture. A nucleic acid extraction from depleted media (0 ng) and 27 nM final CIpB short target E. coli (PC) were used as negative and positive controls respectively. Nanogram (as determined by Nanodrop) amounts of E. coli cell extract per 20 μL reaction are shown. Estimated total number of bacteria cells in a 20 μL reaction is shown in brackets.

FIG. 8 shows fluorogenic aptamer un-nested NASBA produces non-specific products at low concentrations of template independently of primer concentration. A. Ec outer fluorogenic aptamer NASBA reactions were performed for 2 hours, samples were denatured and run into 8% PAGE followed by staining with TO1-Biotin in buffer. E. coli CIpB target concentrations are shown, primers at 250 nM. B. Serial dilutions of Ec outer primer sets in fluorogenic aptamer NASBA reveals non-specific products are not highly dependent on primer concentration and occur under a broad range of primer concentrations. Primer concentrations: 125 nM, 25 nM, 5 nM, 1 nM, 0 M. 25 pM E. coli CIpB target was added or not (Yes/No target) corresponding to light or dark traces respectively.

FIG. 9 shows that only expected RNA sized products are fluorescent in Nested fluorogenic aptamer NASBA in contrast to un-nested NASBA. A. products of the outer NASBA reaction in Nested fluorogenic aptamer NASBA (at 40 min) were loaded into a 8% denaturing PAGE followed by staining with SYBR Safe; Inner NASBA samples shown in figure S5 were collected after 240 min of incubation and were denatured and then run into two 8% PAGE gels followed by staining with either TO1-Biotin (B) or SYBR Safe (C) in buffer.

FIG. 10 shows the alignment of CIpB Short Targets from E. coli (SEQ ID NO: 1) and P. fluorescens (SEQ ID NO: 2). E. coli primer hybridization sites, P. fluorescens hybridization sites. Primer numbering corresponds to that found in Table 1.

FIG. 11 shows a schematic for nucleic acid detection using fluorogenic aptamer template rolling circle amplification. A. Using a two-step ligation-rolling circle amplification (RCA) method, RNA and DNA can be simply and isothermally detected. A template can hybridize to a target, following by its ligation into a circular template. B. This template can now mass produce RNA fluorogenic aptamers by transcription. C. RNA produced as described will yield additional target sites that can be used for further nested ligation, efficiently turning this reaction into an exponential amplification process.

FIG. 12 shows transcription using circular template (left lanes) and linear (right lanes) template results in a long RNA product and short product respectively as a function of time. Time points are 0, 30 s doubling until 64 min.

FIG. 13 shows a ligation with either DNA or RNA target followed by transcription (t=5 min) results in the rapid emergence of fluorogenic aptamer fluorescence.

FIG. 14 shows that 4/5 primer sets targeted against a SARS-CoV-2 sequence were able to successfully detect 1 fM of target RNA in Nested Mango NASBA. Outer reactions for 40 min. P1 and P2 of respective set in Table 2 used as outer reaction, P3 and P4 of respective primer set in Table 2 used as inner reaction.

FIG. 15 show the sensitivity of RNA detection using liquid NASBA kit and detection of SARS-CoV-2 Target 4 RNA in a background of total human RNA. A. Outer reactions performed for 40 min. Human RNA (HNA) added at 5 ng/pL final (gray—positive; black—negative). B. Slopes of the data set shown in (A) signal positives robustly. P1 and P2 (outer) followed by P3 and P4 (inner reaction above) of Primer Set 4 (Table 2).

FIG. 16 shows the sensitivity of SARS-CoV-2 Target 4 RNA detection using liquid (WET) and lyophilized (DRY) NASBA kits. Outer reactions performed for 40 min. P1 and P2 (outer) followed by P3 and P4 (inner reaction above) of Primer Set 4 (Table 2).

FIG. 17 shows that EDTA and heating are not required for low copy SARS-CoV-2 Target 4 RNA detection. 100 aM RNA is equally detected when EDTA (T1) is excluded as well as when the sample is not heated and no EDTA is added (T2). Outer reactions performed for 40 min. WET data from FIGS. 1. P1 and P2 (outer) followed by P3 and P4 (inner reaction above) of Primer Set 4 (Table 2).

FIG. 18 shows the outer reaction time optimization using Lyophilized NASBA kit and SARS-CoV-2 Target 4 RNA. Template concentration was 10 aM. 20 min outer proved to be the shortest time that produced the same robust signal as 40 min outer incubation. P1 and P2 (outer) followed by P3 and P4 (inner reaction above) of Primer Set 4 (Table 2).

FIG. 19 shows the sensitivity of single step Mango NASBA using lyophilized NASBA kit to detect SARS-CoV-2 Target 4 RNA. P5 and P6 of Primer Set 4 (Table 2).

FIG. 20 shows the successful detection of Cultured SARS-CoV-2 RNA using the liquid LS kit. Liquid LS kit was used with the indicated dilutions in a nested fashion. P1 and P2 (outer) followed by P3 and P4 (inner reaction above) of Primer Set 4 (Table 2).

FIG. 21 shows the successful detection of Cultured SARS-CoV-2 RNA using LS lyophilized kit. 1 fM Synthetic SARS-CoV-2 Target 4 RNA was used as a positive control. Detection of cultured viral RNA was tested under different conditions. NOD— 1/20 dilution of outer reaction into inner (usually 1/100 dilution is used); NOR—Only RNA template was heated and no primers; NH—no heating. P1 and P2 (outer) followed by P3 and P4 (inner reaction above) of Primer Set 4 (Table 2).

FIG. 22 show the successful Detection of SARS-CoV-2 RNA from Patient Samples using lyophilized NASBA kit. A. Raw data showing some initial turbidity at low times. B. Plotting the slopes of this data provide unambiguous emergence times. Synthetic corresponds to Target 4 R RNA. P1 and P2 (outer) followed by P3 and P4 (inner reaction above) of Primer Set 4 (Table 2).

FIG. 23 shows that heating of the template by itself or with the primers is not required for the detection of SARS-CoV-2 in patients. Patient sample was subjected to nested Mango NASBA using lyophilized LS kit with and without (NH) prior heating with template. POS indicates patient sample known to have COVID-19. P1 and P2 (outer) followed by P3 and P4 (inner reaction above) of Primer Set 4 (Table 2).

FIG. 24 is a schematic of a fluorogenic aptamer NASBA with an internal control reaction. A liquid container containing a reaction mixture can have oligomers that target an internal control RNA (such as Human 18S ribosomal RNA) as well as the target RNA (such as SARS-CoV-2 RNA).

FIG. 25 is a schematic of an exemplary aptamer-fusion primer.

### DETAILED DESCRIPTION

The present disclosure relates, in part, to the amplification, detection, and/or quantification of nucleic acid molecules.

In some embodiments, the present disclosure provides methods of amplifying target nucleic acid molecules using un-nested and/or nested oligonucleotide primer pairs in isothermal nucleic acid amplification (INA) reactions, such as nucleic acid sequence based amplification (NASBA), rolling circle amplification (RCA), Loop mediated isothermal amplification (LAMP), Recombinase polymerase amplification (RPA), Helicase dependent amplification (HDA), Nicking Enzyme Amplification (NEAR)³⁵, Strand Displacement Amplification (SDA)³⁶, linear and cascade amplification methods, etc.

In some embodiments, the present disclosure further provides methods of detecting target nucleic acid molecules using un-nested and/or nested oligonucleotide primer pairs in isothermal nucleic acid amplification (INA) reactions, such as nucleic acid sequence based amplification (NASBA), rolling circle amplification (RCA), Loop mediated isothermal amplification (LAMP), Recombinase polymerase amplification (RPA), Helicase dependent amplification (HDA), Nicking Enzyme Amplification (NEAR)³⁵, Strand Displacement Amplification (SDA)³⁶, linear and cascade amplification methods, etc.

In some embodiments, the present disclosure further provides methods of quantifying target nucleic acid molecules using un-nested and/or nested oligonucleotide primer pairs in isothermal nucleic acid amplification (INA) reactions, such as nucleic acid sequence based amplification (NASBA), rolling circle amplification (RCA), Loop mediated isothermal amplification (LAMP), Recombinase polymerase amplification (RPA), Helicase dependent amplification (HDA), Nicking Enzyme Amplification (NEAR)³⁵, Strand Displacement Amplification (SDA)³⁶, linear and cascade amplification methods, etc.

In one aspect, the present disclosure provides a nucleic acid molecule, or analog thereof, including: a first nucleic acid sequence, capable of hybridizing to at least a portion of a target nucleic acid sequence, or reverse-complement thereof, and further including an aptamer-encoding template sequence, where the aptamer-encoding template sequence is positioned at the 3′ end of the first nucleic acid sequence; and a second nucleic acid sequence, capable of hybridizing to at least a portion of a target nucleic acid sequence, or reverse-complement thereof, wherein the 5′ end of the second nucleic acid sequence is covalently attached to the 3′ end of the first nucleic acid sequence.

In some embodiments, the 3′ end of the second nucleic acid sequence does not substantially hybridize to the first nucleic acid sequence. In some embodiments, at least the terminal three nucleotides of the 3′ end of the second nucleic acid sequence do not hybridize to the first nucleic acid sequence. In some embodiments, the nucleic acid molecule may include a terminal stem structure, where at least the terminal nucleotide of the 5′ end of the second nucleic acid sequence may be complementary to at least the terminal nucleotide of the 5′ end of the first nucleic acid to form at least a portion of the terminal stem structure. In some embodiments, at least the terminal two or three nucleotides of the 5′ end of the second nucleic acid sequence may be complementary to at least the terminal two or three nucleotides of the 5′ end of the first nucleic acid to form at least a portion of the terminal stem structure.

In another aspect, the present disclosure provides a composition including a first nucleic acid molecule as described herein.

In some embodiments, the composition may further include a second nucleic acid molecule capable of hybridizing to at least a portion of a target nucleic acid sequence, or reverse-complement thereof, and including a first RNA polymerase promoter sequence, where the first and second nucleic acid molecules form a first primer pair capable of amplifying a first sequence of the target nucleic acid sequence.

In some embodiments, the 3′ end of the first nucleic acid molecule may not substantially hybridize to the second nucleic acid molecule or to itself. In some embodiments, the first and second nucleic acid molecules may not substantially hybridize to each other. In some embodiments, the terminal one, two or three bases of the 3′ end of the first nucleic acid molecule may hybridize to the terminal one, two or three bases of the 3′ end of the second nucleic acid molecule. In some embodiments, the 3′ end of the first nucleic acid molecule may be contiguous with the 3′ end of the second nucleic acid molecule when aligned with the sequence of the target nucleic acid.

In some embodiments, the composition as described herein may further include a third nucleic acid molecule and a fourth nucleic acid molecule, where the third and fourth nucleic acid molecules form a second primer pair capable of amplifying a second sequence of the target nucleic acid molecule, where either the third nucleic acid molecule or the fourth nucleic acid molecule may include a second RNA polymerase promoter sequence, and where the second primer pair may hybridize to the target nucleic acid molecule at locations external to that of the first primer pair and may be capable of amplifying the first sequence and the second sequence.

In some embodiments, the second RNA polymerase promoter sequence may transcribe the second sequence of the target nucleic acid molecule in a direction opposite to that of the second nucleic acid molecule. In some embodiments, when the third nucleic acid molecule includes the second RNA polymerase promoter sequence, the fourth nucleic acid molecule includes a second aptamer-encoding sequence, or when the fourth nucleic acid molecule includes the second RNA polymerase promoter sequence, the third nucleic acid molecule includes a second aptamer-encoding sequence. In some embodiments, the 3′ end of the third nucleic acid molecule may not substantially hybridize to the fourth nucleic acid molecule. In some embodiments, the third and fourth nucleic acid molecules may not substantially hybridize to each other. In some embodiments, the 3′ ends of the first, second, third and fourth nucleic acid molecules may not substantially hybridize to each other. In some embodiments, the first, second, third and fourth nucleic acid molecules may not substantially hybridize to each other.

In some embodiments, the composition as described herein may further include a fifth nucleic acid molecule and a sixth nucleic acid molecule, where the fifth and sixth nucleic acid molecules may form a third primer pair capable of amplifying a third sequence of the target nucleic acid molecule, where either the fifth nucleic acid molecule or the sixth nucleic acid molecule may include a third RNA polymerase promoter sequence, where the third primer pair may hybridize to the target nucleic acid molecule at a location external to that of the first and second primer pairs and may be capable of amplifying the first, second and third sequences. In some embodiments, the third RNA polymerase promoter sequence may transcribe the third sequence of the target nucleic acid molecule in the same direction as the second nucleic acid molecule. In some embodiments, when the fifth nucleic acid molecule includes the third RNA polymerase promoter sequence, the fourth nucleic acid molecule includes a third aptamer-encoding sequence, or when the fourth nucleic acid molecule includes the third RNA polymerase promoter sequence, the fifth nucleic acid molecule includes a third aptamer-encoding sequence. In some embodiments, the 3′ end of the fifth nucleic acid molecule may not substantially hybridize to the 3′ end of the fourth nucleic acid molecule. In some embodiments, the fifth and fourth nucleic acid molecules may not substantially hybridize to each other. In some embodiments, the 3′ ends of the first, second, third, fourth, fifth and sixth nucleic acid molecules may not substantially hybridize to each other. In some embodiments, the first, second, third, fourth, fifth and sixth nucleic acid molecules may not substantially hybridize to each other.

In some embodiments, the composition as described herein may include one or more nucleic acid molecules comprising a sequence as set forth in Table 3. In some embodiments, one or more of the nucleic acid molecules may be premixed. In some embodiments, one or more of the nucleic acid molecules may be provided in a liquid. In some embodiments, one or more of the nucleic acid molecules may be lyophilized.

In another aspect, the present disclosure provides a method of amplifying a target nucleic acid sequence, the method including: providing a sample suspected of containing a target nucleic acid molecule; providing a first nucleic acid molecule as described herein; providing a second nucleic acid molecule capable of hybridizing to at least a portion of the target nucleic acid sequence, or complement thereof, and including a first RNA polymerase promoter sequence, where the first and second nucleic acid molecules form a first primer pair capable of amplifying a first sequence of the target nucleic acid sequence; and performing a first amplification reaction including the target nucleic acid molecule and the first primer pair to obtain a first amplification product, where the first amplification product includes the first sequence of the target nucleic acid sequence.

In some embodiments, the 3′ end of the first nucleic acid molecule may not substantially hybridize to the 3′ end of the second nucleic acid molecule. In some embodiments, the first and second nucleic acid molecules may not substantially hybridize to each other. In some embodiments, the terminal one, two or three bases of the 3′ end of the first nucleic acid molecule may hybridize to the terminal one, two or three bases of the 3′ end of the second nucleic acid molecule. In some embodiments, the 3′ end of the first nucleic acid molecule may be contiguous with the 3′ end of the second nucleic acid molecule when aligned with the sequence of the target nucleic acid.

In some embodiments, the method may further include: providing a third nucleic acid molecule and a fourth nucleic acid molecule, where the third and fourth nucleic acid molecules form a second primer pair capable of amplifying a second sequence of the target nucleic acid molecule, where either the third nucleic acid molecule or the fourth nucleic acid molecule includes a second RNA polymerase promoter sequence, where the second primer pair may hybridize to the target nucleic acid molecule at a location external to that of the first primer pair and may be capable of amplifying the first sequence and the second sequence of the target nucleic acid molecule; and performing a second amplification reaction including the first amplification product and the second primer pair to obtain a second amplification product, where the second amplification reaction may be performed prior to the first amplification reaction and where the second amplification product may include the first sequence and the second sequence of the target nucleic acid molecule. In some embodiments, the second RNA polymerase promoter sequence may transcribe the second sequence of the target nucleic acid molecule in a direction opposite to that of the second nucleic acid molecule. In some embodiments, when the third nucleic acid molecule includes the second RNA polymerase promoter sequence, the fourth nucleic acid molecule includes a second aptamer-encoding sequence, or when the fourth nucleic acid molecule includes the second RNA polymerase promoter sequence, the third nucleic acid molecule includes a second aptamer-encoding sequence. In some embodiments, the 3′ end of the third nucleic acid molecule may not substantially hybridize to the 3′ end of the fourth nucleic acid molecule. In some embodiments, the third and fourth nucleic acid molecules may not substantially hybridize to each other. In some embodiments, the 3′ ends of the first, second, third and fourth nucleic acid molecules may not substantially hybridize to each other. In some embodiments, the first, second, third and fourth nucleic acid molecules may not substantially hybridize to each other.A “nucleic acid” or “nucleic acid molecule” is a chain of nucleotides, each of which consists of a nitrogen-containing aromatic base attached to a pentose sugar, which in turn is attached to a phosphate group which connects successive sugar residues by bridging the 5′-hydroxyl group on one sugar to the 3′-hydroxyl group of the next sugar in the chain via phosphodiester bonds. Accordingly, nucleic acids have directionality with a 5′ end and a 3′ end and, by convention, with new nucleotides added to the 3′ end. By convention, nucleic acid “sequences” are written in the 5′ to 3′ direction.

A nucleic acid may be double-stranded or single-stranded. Where single-stranded, the nucleic acid may be the sense strand or the antisense strand. A nucleic acid molecule may be any chain of two or more covalently bonded nucleotides, including naturally occurring or modified nucleotides. By “RNA” is meant a sequence of two or more covalently bonded, naturally occurring or modified ribonucleotides. By “DNA” is meant a sequence of two or more covalently bonded, naturally occurring or modified deoxyribonucleotides. By “cDNA” is meant complementary or copy DNA produced from an RNA template by the action of RNA-dependent DNA polymerase (reverse transcriptase). The terms “nucleic acid” or “nucleic acid molecule” encompass both RNA (plus and minus strands) and DNA, including cDNA, genomic DNA, and synthetic (e.g., chemically synthesized) DNA.

A nucleic acid “analog,” as used herein, is a nucleic acid, including at least one modified nucleotide, that can be amplified by an enzyme, such as a polymerase. In some embodiments, a nucleic acid analog can be amplified by an RNA polymerase, such as T7 RNA polymerase, T3 RNA polymerase, SP6 RNA polymerase and bacterial DNA dependent RNA polymerase. In some embodiments, a nucleic acid analog can incorporate a Locked Nucleic Acid (LNA) nucleotide (Latorra et al., Hum. Mutat. 22:79-85 2003) or Peptide Nucleic acid.

A “modified ribonucleotide” or “modified RNA” includes, without limitation, a RNA with modifications of the 2′-OH group of the ribose (such as 2′-NH2, 2′-fluro, or 2′-O-methyl), and modifications of the nucleobases that do not impede standard Watson-Crick hybridization.

A “modified deoxyribonucleotide” or “modified DNA” includes, without limitation, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine).

By “complementary” or “complementarity” is meant that two nucleic acids, e.g., DNA and/or RNA, contain a sufficient number of nucleotides which are capable of forming Watson-Crick base pairs to produce a region of double-strandedness between the two nucleic acids. Thus, adenine in one strand of DNA and/or RNA pairs with thymine in an opposing complementary DNA strand or with uracil in an opposing complementary RNA strand. It will be understood that each and every nucleotide in a nucleic acid molecule need not form a matched Watson-Crick base pair with a nucleotide in an opposing complementary strand to form a duplex. A nucleic acid is also “complementary” to another nucleic acid if it hybridizes, or is “capable of hybridizing,” with the other nucleic acid.

A “reverse complement” or “complement” sequence, as used herein, is the complementary sequence of a nucleic acid strand, presented 5′ to 3′.

By “capable of hybridizing,” as used herein, is meant that a nucleic acid can base pair with another nucleic acid having a substantially complementary sequence. In some embodiments, by “capable of hybridizing” is meant that a nucleic acid can base pair with another nucleic acid having a substantially complementary sequence under conditions suitable for amplification, such as isothermal amplification. By “substantially” complementary is meant that the base pairing can be partial i.e., not all the nucleotides in one nucleic acid need appropriately base with pair with all the nucleotides in the other nucleic acid and there may be one or more base pairing mismatches between the two nucleic acids. By “a portion of” is meant that the hybridization need not occur along the full length of the nucleic acid(s).

It is to be understood that the stability of the resulting duplex molecule depends upon the extent of the base pairing that occurs, and is affected by parameters such as the degree of complementarity between the two nucleic acids and the degree of stringency of the hybridization conditions. The degree of stringency of hybridization can be affected by parameters such as the temperature, salt concentration, and concentration of organic molecules, such as formamide, and can be determined by methods that are known to those skilled in the art.

By “does not substantially hybridize” is meant that a nucleic acid does not substantially base pair with another nucleic acid under conditions suitable for amplification, such as isothermal amplification. Accordingly, in some embodiments, by “does not substantially hybridize” is meant that a nucleic acid as described herein, such as a the first, second, third, fourth, fifth, or sixth nucleic acids or first, second or third primer pairs do not hybridize with each other or internally. In some embodiments, by “does not substantially hybridize” is meant that the 3′ end, for example, the terminal nine (9) nucleotides, such as the terminal 1, 2, 3, 4, 5, 6, 7, 8, or 9 nucleotides, of a nucleic acid does not base pair within the sequence of any other nucleic acid of the system. In some embodiments, by “does not substantially hybridize” is meant that the 3′ end, for example, the terminal nine (9) nucleotides, such as the terminal 1, 2, 3, 4, 5, 6, 7, 8, or 9 nucleotides, of a nucleic acid does not base pair with the 3′ end, for example, the terminal nine (9) nucleotides, such as the terminal 1, 2, 3, 4, 5, 6, 7, 8, or 9 nucleotides, of another nucleic acid.

By “amplification” is meant a process by which additional copies of a nucleic acid sequence are produced. Nucleic acid amplification processes are known in the art and can include, without limitation, polymerase chain reaction (PCR), such as methylation sensitive PCR, nested-PCR, cold-PCR, digital PCR, droplet digital PCR, ICE-cold-PCR, multiplex PCR (mPCR), real-time or quantitative PCR (qPCR), reverse transcriptase (RT)-PCR, or quantitative reverse transcriptase (RT)-PCR.

In some embodiments, the “amplification” process may be isothermal i.e., where amplification is performed at a constant temperature. Isothermal amplification of nucleic acids (INA) can include, without limitation, Nucleic acid sequence based amplification (NASBA), Rolling Circle Amplification (RCA), Loop mediated isothermal amplification (LAMP), Recombinase polymerase amplification (RPA), Helicase dependent amplification (HDA), Nicking Enzyme Amplification (NEAR), Strand Displacement Amplification (SDA), or linear and cascade amplification methods.

In some embodiments, a suitable isothermal amplification method, such as an isothermal amplification method that includes an RNA intermediate, can be used as exemplified by the NASBA or TMA methods as described herein or known in the art. Accordingly, in some embodiments, RNA producing isothermal amplification methods can produce an antisense sequence using a reverse primer that includes an RNA polymerase promoter sequence such as T7, T3 or SP6. This can produce an RNA output which is the reverse complement of the input sequence. Accordingly, in some embodiments, when nesting a reaction, the inner nested reaction requires an RNA polymerase promoter sequence on the opposite primer. In some embodiments, RNA producing isothermal amplification methods can be used together with a fluorogenic aptamer template(s), as described herein or known in the art.

In some embodiments, other isothermal amplification methods can be readily adapted and used as described herein. For example, RCA can be adapted by transcribing RNA off a DNA circle, as described herein.

In alternative embodiments, other DNA-based isothermal methods, such as LAMP, RPA, NEAR, HDA or SDA can be similarly adapted by the addition of an RNA polymerase promoter to a DNA oligonucleotide, in accordance with the isothermal amplification method to be used, whereby RNA transcription serves to report the DNA amplification products produced by the isothermal method. In some embodiments, HDA, RPA, or NEAR primers can be modified to have RNA polymerase promoters and enzyme(s) and fluorogenic aptamer template(s), enabling RNA aptamer production as a reporter of successful amplification. In some embodiments, HDA, RPA, or NEAR primers can be modified to have DNA fluorogenic aptamer template(s), enabling DNA aptamer production as a reporter of successful amplification.

In some embodiments, conditions suitable for amplification may be conditions suitable for PCR, as known in the art. In some embodiments, conditions suitable for isothermal amplification may be conditions suitable for the specific isothermal amplification method of choice, such as NASBA, RCA, LAMP, HAD, SDA, etc., as described herein or known in the art.

For example, for NASBA, conditions suitable for amplification may include amplification of an RNA product isothermally at about 41° C. first using a primer containing a RNA polymerase promoter (e.g., T7 promoter), as described herein or known in the art, that hybridizes to a target nucleic acid, e.g., RNA and is extended by a reverse transcriptase (RT). RNase H then degrades the hybridized RNA to leave the bare cDNA. Next, a second primer, as described herein or known in the art, hybridizes to the cDNA and is extended by the RT to the end of the initial hybridizing primer, producing a dsDNA containing a T7 promoter. The RNA polymerase then transcribes an RNA encoded between the regions where the primers originally annealed used. As multiple copies of RNA are made, free primer can continue to hybridize, be extended, and produce more template, resulting in exponential amplification of the DNA template and RNA product.

In some embodiments, amplification parameters, for example, for NASBA using a Mango aptamer, may include one or more of the following:
a. No heating of the RNA sample prior to performing the NASBA reaction;
b. No EDTA;
c. Shorter reaction time, for example, about 20 minutes;
d. About 20 fold dilution from the outer to the inner reaction, in the case of a nested reaction; and/or
e. Lyophilization of reagents.

In general, isothermal reactions consist of a single set of isothermal amplification nucleic acids specified as the set required to complete the exponential amplification process. By contrast, in some embodiments, the present disclosure provides isothermal amplification reactions that can be multiplexed, as described herein. For example, “n,” where n can be 1, 2, or 3 or higher, sets of isothermal amplification primers or “primer pairs” can be generated. The primer sets or pairs can be distinct, for each target nucleic acid to be amplified. This allows the amplification, detection and/or quantification of “n” target nucleic acids by “multiplexing,” i.e., simultaneous detection of multiple target nucleic acids within the same reaction, which can permit important internal control and validation. In this example, the appropriate number of primer sets is provided, for example, in a reaction mixture. For example, two primer sets may be provided for preferentially amplifying two target nucleic acid sequences, three primer sets may be provided for preferentially amplifying three target nucleic acid sequences and so on. In some embodiments, detection may be based on the unique sequences of each primer set used. In some embodiments, fluorogenic detection may be used in multiplexed amplification methods, as described herein or known in the art. In some embodiments, different fluorophores having, for example, distinct emission spectra may be used. In some embodiments, orthogonal two-colour or three-colour fluorogenic aptamers and their corresponding ligands may be used, as described herein.

In some embodiments, isothermal amplification reactions can be “nested,” as described herein. In such embodiments, dilution of the amplification product prior to performing a subsequent amplification with, for example, nested primer pairs may substantially improve sensitivity and specificity and reduce amplification artifacts. In some embodiments, nested amplification reactions, for example, nested isothermal amplification reactions can be “multiplexed.” In some embodiments, such nested and multiplexed amplification reactions can be used in conjunction with fluorogenic detection methods.

In general, the amplification reaction is performed in a reaction mixture. By “reaction mixture,” as used herein, is meant a composition including the relevant components to allow an amplification reaction to be performed. An exemplary reaction mixture can include, without limitation, a nucleic acid sample, primer pairs, and a suitable enzyme, such as a polymerase. One of skill in the art will appreciate that the reaction mixture may also include other components such as buffers, stabilisers, templates, nucleotides and the like and that these components may be dictated by the amplification reaction being performed.

It is to be understood that amplification parameters, such as nucleotide concentration, nucleic acid polymerases used for the amplification, buffer composition, number of amplification cycles, temperatures during the cycles, can be optimized as described herein or known in the art.

A “target nucleic acid,” “target nucleic acid molecule” or “target nucleic acid sequence” refers to any nucleic acid that can be amplified, for example, as described herein. In some embodiments, a target nucleic acid can be detected. In some embodiments, a target nucleic acid can be quantified.

It is to be understood that the target nucleic acid can be of any size, as long as it can be amplified using, for example, a polymerase, such as an RNA polymerase. In some embodiments, a target nucleic acid may be about 100 to about 10,000 nucleotides long, or any value in between. In another example, the target nucleic acid may be about 100 to about 5,000 nucleotides long, or any value in between. In another example, the target nucleic acid may be about 100 to about 3,000 nucleotides long, or any value in between. In another example, the target nucleic acid may be about 100 to about 2,000 nucleotides long, or any value in between. In another example, the target nucleic acid may be about 100 to about 1,000 nucleotides long, or any value in between. In another example, the target nucleic acid may be about 100 to about 500 nucleotides long, or any value in between.

Target nucleic acid molecules include, without limitation, RNA or DNA, for example, chromosomal DNA, mitochondrial DNA, messenger RNA, ribosomal RNA, transfer RNA, viral RNA and extrachromosomal DNA, such as virulence plasmids. The target nucleic acid molecule may be present in a sample, such as a biological sample, a forensic sample, a synthetic sample or an environmental sample.

An “aptamer,” as used herein, refers to a nucleic acid molecule that can bind a ligand, such as a peptide, small molecule (e.g., an antibiotic), carbohydrate, etc., with high selectivity and specificity i.e., “specifically bind” the ligand. In some embodiments, an aptamer can include a modified nucleotide that can be amplified by an enzyme, such as a polymerase. In some embodiments, an aptamer can include a modified nucleotide that can be amplified by an RNA polymerase, such as T7 RNA polymerase, T3 RNA polymerase, SP6 RNA polymerase, or bacterial or eukaryotic RNA polymerase. It is to be understood that an RNA polymerase may be obtained from any suitable source, such as a virus, bacteriophage, bacteria, or eukaryote such as plant or animal, In some embodiments, an aptamer may be a single-stranded (ss) nucleic acid (e.g., ssRNA or ssDNA). A single-stranded nucleic acid aptamer can assume a variety of shapes including helices and single-stranded loops. Accordingly, aptamer-ligand binding can be determined by tertiary, rather than primary, structure. In some embodiments, an aptamer includes a terminal stem structure i.e., a duplex structure comprising the 3′ and 5′ ends of the aptamer. In some embodiments, the terminal stem structure may be as short as 2 bp and can be arbitrarily long. In some embodiments, the terminal stem structure may be about 6 bp to about 8 bp.

An “aptamer-encoding template sequence,” as used herein, is the nucleic acid sequence that is the reverse complement of a nucleic acid aptamer sequence.

In some embodiments, the ligand can be a signal-generating ligand that, for example, generates a fluorescent signal (e.g., from a fluorophore) or a colorimetric signal. A fluorogenic RNA aptamer sequence can be selected using in vitro selection to optimize both the fluorescent enhancement of the fluorogenic aptamer system (F_{E}) and the K_{D} of the aptamer-fluorophore interaction. Examples of fluorophore binding aptamers include, without limitation, Mango, Pepper, Broccoli, Corn, Spinach and Spinach2 (Strack et al, Nature Methods 2013, 10: 1219-1224), Carrot and Radish (Paige et al, Science 2011, 333:642-646), RT aptamer (Sato et al., Angew. Chem. Int. Ed. 2014, 54: 1855-1858), hemin-binding G-quadruplex DNA and RNA aptamers, or malachite green binding aptamer (Babendure et al, J. Am. Chem. Soc. 2003). Fluorophores include, without limitation, infrared (IR) dyes, Dyomics dyes, phycoerythrine, cascade blue, Oregon green 488, pacific blue, rhodamine derivatives such as rhodamine green, 5(6)-carboxyfluorescein, cyanine dyes (i.e., Cy2, Cy3, Cy 3.5, Cy5, Cy5.5, Cy 7) (diethyl-amino)coumarin, fluorescein (i.e., FITC), tetramethylrhodamine, lissamine, Texas Red, AMCA, TRITC, bodipy dyes, or Alexa dyes.

A “Mango” or “Mango aptamer” refers to an RNA aptamer. The RNA Mango aptamer series have extremely high contrast making them useful in vitro fluorescent reporters. These aptamers have nanomolar binding affinity to a thiazole orange-based ligand (TO1-Biotin) that is capable of becoming up to 4,000 times brighter upon binding an RNA Mango aptamer. RNA Mango aptamers Mango II, III, and IV are highly resistant to the magnesium ion concentrations found in in vitro assays and also work in a range of monovalent metal ion concentrations. Mango III has also been recently improved by structure guided engineering to become even brighter.

A “Broccoli” or “Broccoli aptamer” refers to a 49-nt fluorescent RNA aptamer (see, for example, Filonov et al., J. Am. Chem. Soc. 2014, 136(46): 16299-16308) that confers fluorescence to a target analyte (e.g., target RNA) of interest via activation of the bound fluorophore DFHBI or a DFHBI-derived fluorophore such as (Z)-4-(3,5-difluoro-4-hydroxybenzylidene)-2-methyl-I-(2,2,2-trifluoroethyl)-IH-imidazol-5(4H)-one) (DFHBI-IT) as described by Song et al., J. Am. Chem. Soc. 2014, 136: 1198.

An aptamer “specifically binds” a ligand when it recognises and binds the ligand, for example, a flurophore, but does not substantially recognise and bind other molecules in a sample. In some embodiments, an aptamer can have, for example, an affinity for the ligand which is at least 10, 100, 1000 or 10,000 times greater than the affinity of the aptamer for another reference molecule in a sample. In some embodiments an aptamer sequence can have a ligand binding dissociation constant (K_{D}) between about 0.01 nM and about 100 nM, or any value in between such as 0.2 nM. In some embodiments a fluorogenic aptamer sequence can have a fluorophore binding dissociation constant (K_{D}) between about 0.01 nM and about 100 nM, or any value in between, such as 0.2 nM. In some embodiments a fluorogenic aptamer encoding sequence can have a fluorophore binding dissociation constant (K_{D}) between about 0.01 nM and about 100 nM, or any value in between, such as 0.2 nM. It is to be understood that selection of a suitable aptamer, such as a fluorescent RNA aptamer-fluorophore complex for use as described herein, can depend on a variety of parameters depending on the characteristics of the aptamer such as binding affinity, brightness, secondary structure, amenability to sequence modifications, etc.

In some embodiments, orthogonal two-colour fluorogenic aptamers and ligands may be used, as described herein. Two fluorogenic ligand binding aptamers are orthogonal to each other with respect to binding if the first aptamer specifically binds its ligand and the second aptamer binds specifically to its ligand. It is to be understood that some overlap in binding may occur. In some embodiments, each fluorogenic ligand has an emission spectrum that is distinct from the other to allow robust two colour quantification of each aptamer concentration. In some embodiments, orthogonal three-colour fluorogenic aptamers and ligands may be used based on the same concept. This concept of orthogonality is easily extended to three-colour imaging or higher, as would be appreciated by one of skill in the art.

The term “primer” refers to a relatively short nucleic acid sequence that is complementary to at least a portion of a target nucleic acid molecule or sequence. It is to be understood that a primer can in addition be complementary to the reverse complement of at least a portion of a target nucleic acid molecule or sequence.

In some embodiments, a primer has a “degenerate” sequence i.e., the nucleic acid sequence is a composition of sequences that have different nucleotides at the same position such that the primer is a mixture of different sequences that can hybridize to multiple, different target nucleic acids. In other words, a degenerate sequence can be complementary to a plurality of target nucleic acid sequences.

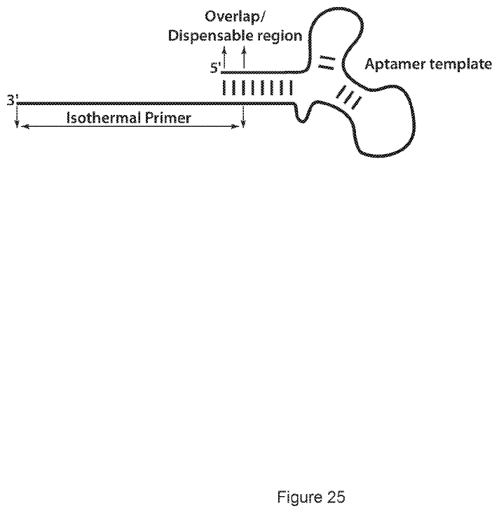

In some embodiments, a primer may include a first nucleic acid sequence, capable of hybridizing to at least a portion of a target nucleic acid sequence, or complement thereof, as well as an aptamer-encoding template sequence, where the aptamer-encoding template sequence is positioned at the 3′ end of the first nucleic acid sequence; and a second nucleic acid sequence, capable of hybridizing to at least a portion of a target nucleic acid sequence, or complement thereof, where the 5′ end of the second nucleic acid sequence is covalently attached to the 3′ end of the first nucleic acid sequence, and where the 3′ end of the second nucleic acid sequence does not substantially hybridize to the first nucleic acid sequence. Such a primer may be referred to herein as an “aptamer-fusion primer.” In some embodiments, at least the terminal nucleotide of the 5′ end of the second nucleic acid sequence may be complementary to at least the terminal nucleotide of the 5′ end of the first nucleic acid to form at least a portion of the terminal stem structure. In some embodiments, at least the terminal two or three nucleotides of the 5′ end of the second nucleic acid sequence may be complementary to at least the terminal two or three nucleotides of the 5′ end of the first nucleic acid to form at least a portion of the terminal stem structure. A schematic representation of an exemplary aptamer-fusion primer is shown in FIG. 25.

In some embodiments, the first nucleic acid sequence may be about 20 to about 100 nucleotides in length, or any value in between, such as about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In some embodiments, the first nucleic acid sequence may be more than about 100 nucleotides in length, such as 200 nt long.

In some embodiments, the second nucleic acid sequence may be about 15 to about 100 nucleotides in length, or any value in between, such as about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

In some embodiments, the aptamer-fusion primer may include a linker sequence between the first nucleic acid sequence and the second nucleic acid sequence. The linker sequence may be 0 to about 65-nt nucleotides in length, or any value in between, such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65.

It is to be understood that similar considerations apply to the third, fourth, fifth, or sixth nucleic acids, or additional nucleic acids, depending on whether they are designed to form aptamer fusion primers or include a polymerase promoter, as would be understood by one of skill in the art or described herein.

By “primer pair” is meant two optimally designed nucleic acid sequences, as described herein, which can serve to prime an amplification reaction, such as an isothermal amplification reaction, where the nucleic acid sequences anneal to complementary sequences on the target nucleic acid sequence.

A “sample” can be any organ, tissue, bodily fluid, cell, or cell extract isolated or extracted from an organism or any material that contains, potentially contains, or is suspected of containing, nucleic acid from an organism. For example, a sample from an animal, such as a mammal, can include, without limitation, cells or tissue (e.g., from a biopsy or autopsy) from bone, brain, breast, colon, muscle, nerve, ovary, prostate, retina, skin, skeletal muscle, intestine, testes, heart, liver, lung, kidney, stomach, pancreas, uterus, adrenal gland, tonsil, spleen, soft tissue, peripheral blood, whole blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, semen, mammalian colostrum, mucosal cells, milk, urine, feces, stool, lacrimal fluid, saliva, placental extracts, amniotic fluid, a cryoprecipitate, a cryosupernatant, a cell lysate, mammalian cell culture or culture medium, products of fermentation, ascitic fluid, proteins present in blood cells, tracheal aspirate, nasal aspirate, oropharyngeal swab, or any other specimen, or any extract thereof, obtained from an organism (e,g, human or animal), test subject, or experimental animal. A sample may also include, without limitation, products produced in cell culture by normal or transformed cells (e.g., via recombinant DNA or monoclonal antibody technology). A sample may also include, without limitation, any organ, tissue, cell, or cell extract isolated from a non-mammalian animal, such as a bird, a fish, an insect or a worm. In another example, the sample can be a fungal sample. In another example, the sample can be obtained from a plant.

A “sample” may also be a cell or cell line created under experimental conditions, that is not directly isolated from an organism. A sample can be using standard techniques, such as brushes, swabs, spatulae, rinse/wash fluids, punch biopsy devices, puncture of cavities with needles or surgical instrumentation. Tissue or organ samples may be obtained from any tissue or organ by, e.g., biopsy or other surgical procedures. Separated cells may be obtained from the body fluids or the tissues or organs by separating techniques such as filtration, centrifugation or cell sorting.

In some embodiments, a “sample” can be collected or extracted, without limitation, from the environment, such as from air, water or soil; from material intended for human or animal consumption, such as meat, fish, dairy, or feed; from cosmetics, agricultural products, plastic and packaging materials, paper, clothing fibers, metal surfaces, etc.; A sample can also be cell-free, artificially derived or synthesized, for example, be a synthetic construct, such as a synthetic nucleic acid. A sample may be in liquid form including, without limitation, the traditional definition of liquid as well as colloids, suspensions, slurries, and dispersions.

Methods of obtaining or extracting a nucleic acid, such as DNA or RNA are well known in the art and include, without limitation, RNA extraction spin columns, phenyl/chloroform-based extraction methods, etc. In some embodiments, the nucleic acid can be a DNA or RNA target that can be extracted using automated techniques and equipment.

A “control” includes a sample obtained for use in determining base-line expression or activity. control also includes a previously established standard or reference. Accordingly, any test or assay conducted according to the invention may be compared with the established standard or reference and it may not be necessary to obtain a control sample for comparison each time.

An organism can be, without limitation, a virus, a microorganism, mycoplasma, fungus, animal (e.g., a mammal), a plant, a bacterium, an alga, a parasite, a fungus, or a protozoan. In some embodiments, the animal may be a human, non-human primate, rat, mouse, cow, horse, pig, sheep, goat, dog, cat, etc. The organism may be a clinical patient, a clinical trial volunteer, an experimental animal, a domesticated animal, etc.

Exemplary plants include monocotyledons, dicotyledons and the conifers. For example, plants can include, but are not limited to, cereals, grapes, beet, pomes, stone fruit and soft fruit; leguminous plants, oil plants, cucumber plants, fibre plants, citrus fruit, vegetables, lauraceae and plants such as maize, tobacco, nuts, coffee, sugar cane, tea, vines, hops, turf, bananas, natural rubber plants or ornamentals.

Examples of fungi include without limitation yeasts, Aspergillus spp.; Blastomyces dermatitidis; Candida; Coccidioides immitis; Coccidioides posadasii; Cryptococcus neoformans; Histoplasma capsulatum; Pneumocystis species.

Maize rust; Rice blast; Rice brown spot disease; Rye blast; Sporothrix schenckii; wheat fungus, etc.

Examples of protozoa and worms include without limitation parasitic protozoa and worms, such as: Acanthamoeba and other free-living amoebae; Anisakis sp. and other related worms; Cryptosporidium parvum; Cyclospora cayetanensis; Diphyllobothrium spp.; Entamoeba histolytica; Eustrongylides sp.; Giardia lamblia; Nanophyetus spp.; Shistosoma spp.; Toxoplasma gondii; or Trichinella.

Examples of analytes include without limitation allergens such as plant pollen and wheat gluten.

In some embodiments, the organism may be pathogenic, such as a bacterial or viral pathogen.

Examples of bacterial pathogens include, without limitation, Aeromonas hydrophila; Bacillus anthracis; Bacillus cereus; Botulinum neurotoxin producing species of Clostridium; Brucella abortus; Brucella melitensis; Brucella suis; Burkholderia mallei (formally Pseudomonas mallei); Burkholderia pseudomallei (formerly Pseudomonas pseudomallei); Campylobacter jejuni; Chlamydia psittaci; Clostridium botulinum; Clostridium botulinum; Clostridium perfringens; Coccidioides immitis; Coccidioides posadasii; Cowdria ruminantium; Coxiella burnetii; Enterovirulent Escherichia coli group (EEC Group) such as Escherichia coli-enterotoxigenic (ETEC), Escherichia coli-enteropathogenic (EPEC), Escherichia coli-O157:H7 enterohemorrhagic (EHEC), and Escherichia coli-enteroinvasive (EIEC); Ehrlichia spp. such as Ehrlichia chaffeensis; Francisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacterium tuberculosis; Mycoplasma capricolum; Mycoplasma mycoides ssp mycoides; Rickettsia prowazekii; Rickettsia rickettsii; Salmonella spp.; Schlerophthora rayssiae varzeae; Shigella spp.; Staphylococcus aureus; Streptococcus; Synchytrium endobioticum; Vibrio cholerae non-O1; Vibrio cholerae 01; Vibrio parahaemolyticus and other Vibrios; Vibrio vulnificus; Xanthomonas oryzae; Xylella fastidiosa; Yersinia enterocolitica and Yersinia pseudotuberculosis; or Yersinia pestis.

Examples of viral pathogens include without limitation single stranded RNA viruses, single stranded DNA viruses, double-stranded RNA viruses, or double-stranded DNA viruses. In some embodiments, pathogenic viruses include, without limitation, African horse sickness virus; African swine fever virus; Akabane virus; Bhanja virus; Caliciviruses (e.g., human enteric viruses such as norovirus and sapovirus), Cercopithecine herpesvirus 1; Chikungunya virus; Classical swine fever virus; coronaviruses (e.g., Severe Acute Respiratory Syndrome (SARS), Middle East Respiratory Syndrome (MERS), Severe Acute Respiratory Syndrome coronavirus 2 (SARS-CoV-2)); Dengue viruses such as serotypes 1 (DENV1) and 3 (DENV3), and related viruses such as the chikungunya virus (CHIKV); Dugbe virus; Ebola viruses; Encephalitic viruses such as Eastern equine encephalitis virus, Japanese encephalitis virus, Murray Valley encephalitis, and Venezuelan equine encephalitis virus; Equine morbillivirus; flaviruses, Flexal virus; Foot and mouth disease virus; Germiston virus; Goat pox virus; Hantaan or other Hanta viruses; Hendra virus; human immunodeficiency virus (HIV); influenza viruses (e.g., H1 N1, H5N1, Avian influenza virus); Lassa fever virus; Louping ill virus; Lymphocytic choriomeningitis virus; Poliovirus; Potato virus; pox viruses; South American hemorrhagic fever viruses; Variola major virus (Smallpox virus); Vesicular stomatitis virus; West Nile virus; Yellow fever virus; human-pathogenic flaviviruses such Zika virus, etc.

In some embodiments, the target nucleic acid can be detected simultaneously or subsequently to the amplification. The term “detect” or “detection” as used herein indicates the determination of the existence, presence or fact of a target nucleic acid or signal in a sample or a reaction mixture.

In some embodiments, the target nucleic acid can be quantified simultaneously or subsequently to the amplification and detection. The quantification may include, without limitation, the measurement of quantity or amount of the target or signal (also referred as quantitation), which includes but is not limited to any analysis designed to determine the amounts or proportions of the target or signal. Detection is “qualitative” when it refers, relates to, or involves identification of a quality or kind of the target or signal in terms of relative abundance to another target or signal, which is not quantified. An “optical detection” indicates detection performed through visually detectable signals: fluorescence, spectra, or images from a target of interest or a probe attached to the target.

In some embodiments, the methods of the present disclosure can be incorporated into methods of diagnosis by amplifying, detecting and/or quantifying the level of a target sequence indicative of a disease, disorder or pathological condition.

In some embodiments, the methods of the present disclosure can be incorporated into methods of forensic or environmental analysis by amplifying, detecting and/or quantifying the level of a target sequence indicative of a crime or of contamination.

In various embodiments, the design of the primers can be optimized. In some embodiments, the un-nested and/or nested oligonucleotide primer pairs can have decreased opportunity for primer dimer formation, as well as decreased opportunity for non-specific hybridization to the target nucleic acid molecule. Accordingly, in some embodiments, the un-nested and/or nested oligonucleotide primer pairs can have one or more of the following characteristics.

1. the primer pairs can be designed to have the lowest potential hybridization with each other. For example, in some embodiments, the primer pairs may be designed to have less than or equal to 3 nucleotides capable of hybridization to each other. In alternative embodiments, the primer pairs should not hybridize to one another.

2. the primers can have as few as possible alternative target sites to the nucleic acid (e.g. RNA) sequence of interest. In some embodiments, the primers can be designed to preclude hybridization at their 3′ ends to either undesired target sites or to other primer sequences of the design. In some embodiments, the 3′ ends should not allow the primers self-extension (by for example fold back hybridization).

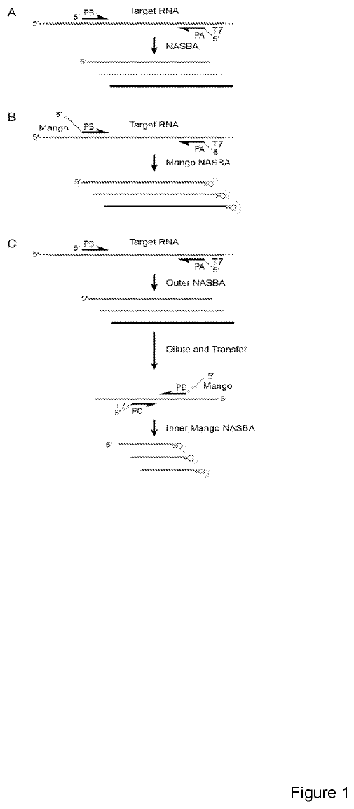

3. For un-nested situations: A DNA primer, which can, at the isothermal temperature of the utilization, hybridize to the 3′ region of an RNA of interest by using the 3′ sequence of the DNA primer. In some embodiments, the 3′ terminus of this primer can be able to fully hybridize to the RNA of interest by at least 1 to 3 nt of terminal sequence. In some embodiments, at the 5′ of this hybridization region, which may or may not be fully hybridized to the RNA of interest, a RNA polymerase promoter sequence can be included in the primer sequence (for example that of T7, T3 or SP6) (PA, FIG. 1A & B). Hybridization of the PA primer to the RNA target can be estimated by thermodynamic calculations to be stable in the salt and buffer conditions used for the isothermal amplification system, using standard techniques. In some embodiments, there may be 15-30 bp of hybridization, but is not limited to such.

4. A second primer (PB, FIG. 1A&B) able to hybridize to the reverse complement of the RNA target sequence and designed otherwise similarly to primer PA, can hybridize to the RNA's reverse complement sequence found 5′ to the location of hybridization of the PA primer. Should a fluorogenic aptamer reporter be included in the design, the reverse complement of such an aptamer sequence can be included within the 5′ region of the PB primer (PB, FIG. 1B). In some embodiments, the hybridization sites for primers PA and PB may be designed to be as close together as possible for most efficient isothermal amplification. In some embodiments, the 3′ ends of the primers do not overlap. In alternative embodiments the 3′ ends of the primers may be within 500-nt of each other so as to permit effective nesting of the inner primer pair.

5. For nested primer designs, an ‘outer primer pair can be designed as for un-nested primers described herein, with the following additional criteria: The distance between the PA and PB outer primers can be sufficient to allow the inner primers to hybridize between the 3’ ends of the outer PA and PB primers. Primer PB in such cases can be designed to include a fluorogenic aptamer sequence or in some utilizations no aptamer sequence is included (for example Mango FIG. 1C). The inner primers PC and PD (FIG. 1C) can be designed to hybridize by the same criteria as PA and PB respectively. Note that these primers are amplifying an RNA that is the reverse complement of the original RNA target and that PC can include a promoter sequence as discussed for PA herein and that primer PD can either include or not include a fluorogenic aptamer sequence. In some embodiments this is not required owing to leakage of the RNA polymerases involved. In some embodiments, the hybridization regions for PC and PD can partially overlap with the PA and PB hybridization regions to for example minimize the potential for artefactual sequence amplification.

In some embodiments, where inner primer PD includes a fluorogenic aptamer (e.g. Mango, FIG. 1C) outer primer PB does not include a fluorogenic aptamer. In some embodiments, where a fluorogenic aptamer is included on PB, a distinct fluorogenic aptamer sequence can be included on the inner primer PD. In some embodiments, the distinct aptamer can have spectrally distinct properties to the fluorogenic aptamer found on outer primer PB (for example Pepper, Broccoli or Corn aptamers on PB and Mango aptamer on PD). In some embodiments, the fluorogenic aptamers can be fully functional in the isothermal buffer of the isothermal amplification system (for example RNA Mango aptamers, which are broadly tolerant of salt and pH and chemical conditions).

In some embodiments, use of nested oligonucleotide primer pairs can increase the sensitivity and/or specificity of the INA. In some embodiments, use of the nested oligonucleotide primer pairs as described herein results in a sensitivity of at least 10⁻¹⁹ M to about 10⁻⁶ M concentrations. In some embodiments, use of the nested oligonucleotide primer pairs as described herein results in a sensitivity of attomolar 10⁻¹⁸ M concentration.

In some embodiments, the INA detection methods using the nested oligonucleotide primer pairs as described herein can be used in, without limitation, fluorogenic aptamers such as Mango etc, molecular beacons, nonspecific NA intercalation fluorescent stains, and/or gel-based detection methodologies.

In some embodiments, the INA detection methods using the nested oligonucleotide primer pairs as described herein are insensitive or less sensitive to nonspecific amplification artifacts (off target effects).

In some embodiments, the INA detection methods using the nested oligonucleotide primer pairs as described herein are fast and convenient and can be arranged to directly give real time fluorescent read outs.

In some embodiments, the nested oligonucleotide primer pairs as described herein can include fluorogenic aptamer sequences, such as but not limited to RNA Mango. Introduction of a fluorogenic ligand for its corresponding aptamer can result in the creation of a real-time fluorescent reporter system. Accordingly, INA detection methods using oligonucleotide primers that include fluorogenic aptamer sequences (INAF) can enable real time isothermal NA detection. In some embodiments, INAF methods can be used for the detection of relatively high abundance nucleic acid target sequences, such as but not limited to template concentrations in the microM to picoM concentration range.

In some embodiments, the primers and/or targets, in accordance with the present disclosure may include without limitation the nucleic acid sequences set forth herein, such as in Table 3 or sequences having at least 90% to 99.9% similarity, or any value in between, such as at least 91%, 92%, 93%, 94%, 95%, 96%, 97%, 98%, or 99% similarity, to the sequences of Table 3. In some embodiments, the primers and/or targets, in accordance with the present disclosure may include without limitation the sequences set forth in Table 3 or sequences having at least 90% to 99.9% identity, or any value in between, such as at least 91%, 92%, 93%, 94%, 95%, 96%, 97%, 98%, or 99% identity, to the sequences of Table 3.

In some embodiments, INAF methods can be used for the detection of low concentration or low abundance nucleic acid target sequences such as, but not limited to, attomolar, 10⁻¹⁸ M or 1 NA molecule per microliter sample. Such methods, termed Isothermal Nested Fluorogenic Amplification and Detection (INFAD), include: an initial outer primer isothermal amplification step, followed by a subsequent nested inner isothermal reaction using primers containing the fluorogenic aptamer tagged primers. It is to be understood the method is not limited to a single nesting event, for example 1 or more nesting events may take place. Without being bound to any particular hypothesis, a single nesting can preclude many amplification artifacts. In some embodiments, a second or additional nesting may improve sensitivity still further.

The INFAD inner and outer primer pairs are modified to maximize sensitivity including but not limited to methodologies where primers are arranged such that nucleic acid fluorogenic aptamers are made at the end of the innermost exponential isothermal amplification cycle and not at earlier steps in the amplification process.

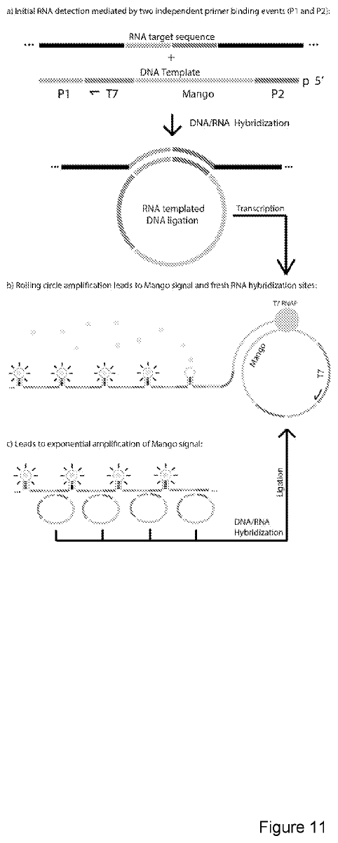

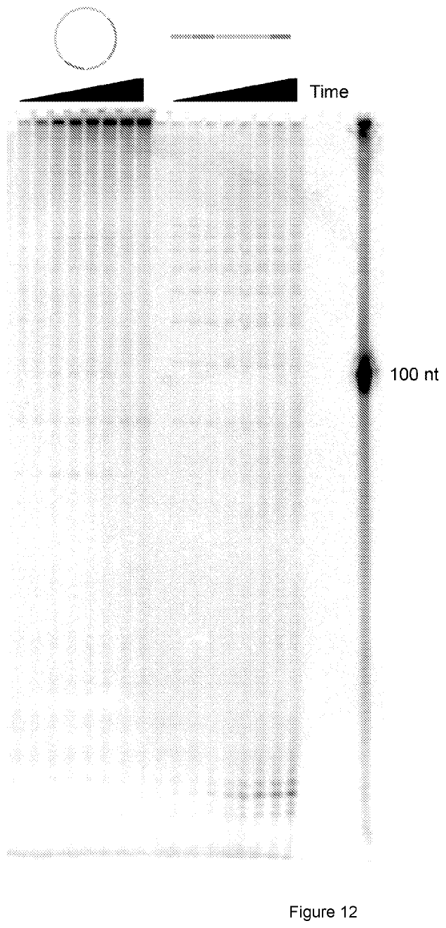

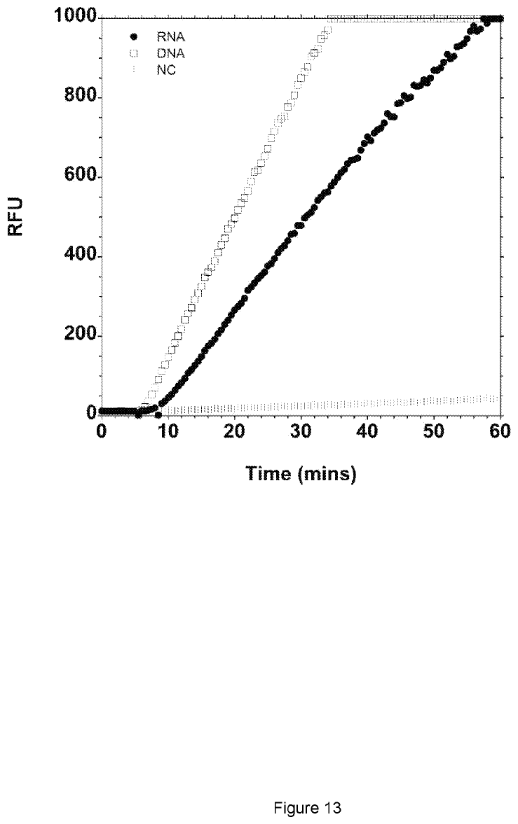

For un-nested RCA, DNA or RNA target sequences can be detected. In some embodiments, a linear DNA oligonucleotide should contain a fluorogenic aptamer (e.g. Mango, FIG. 11), a DNA promoter sequence (e.g. T7, T3, SP6) orientated to allow the production of the aptamer sequence and should also contain the ability to be ligated. This can be performed for example by adding a 5′ phosphate to the DNA oligo and using T4 DNA ligase in the implementation. The 5′ and 3′ regions of this oligonucleotide (“linear RCA template” is used but not limited to Table 1 or 3) should contain hybridization regions to the DNA or RNA target of interest (“target RCA splint” is used but not limited to, Table 1 or 3) so as to allow hybridization of the DNA oligonucleotide such that the 5′ and 3′ termini are immediately proximal so as to allow ligation (which could be enzymatic or chemical if for example an imidazole activation of the 5′ phosphate was implemented). Addition of a top strand promoter sequence (“T7 promoter complement” is used, but not limited to, Table 1 or 3) after or before ligation then allows transcription by for example T7 or SP6 polymerase (T7, FIG. 11, 12). Amplification resulting from DNA or RNA targets can be implemented by the creation of long repetitive RNA sequences having the reverse complement of the DNA oligonucleotide sequence (FIGS. 11-13). The system holds exponential amplification potential, as each turn of the circle by RNA polymerase produces a new ligation target site that can promote further circle production.

Nesting of RCA can be simply envisioned by hybridization of the 5′ and 3′ oligonucleotide as just described to include a gap sequence between the hybridization sites of the oligonucleotide. This implies that the length of the oligonucleotide is sufficient to allow such a gap, which can be imagined to be from 20 to 50 nt of RNA target sequence. By addition of a nonstrand displacing RT enzyme this gap can be filled allowing ligation as just described. The resulting repetitive sequence will not contain a region of RNA sequence complementary to the target RNA sequence found between the two oligonucleotide hybridization sites. Thus, a second amplification cycle can be designed with a DNA oligonucleotide sequence now designed to hybridize to this inner region of sequence. In some embodiments, it is efficacious to only include a fluorogenic aptamer on the inner primer oligonucleotide or two have distinct fluorogenic aptamers on the ‘outer’ and ‘inner’ oligonucleotides of the design.

In some embodiments, INFAD methods can include a two-color aptamer fluorophore systems where nucleic acid aptamerl binds specifically fluorophore1 (A1:F1 fluorogenic complex) and aptamer2 binds specifically to fluorophore2 (A2:F2 fluorogenic complex) where the fluorescent emission from A1:F1 and A2:F2 is distinguishable using fluorimeters and enables a sensitive two channel INFAD system. Such systems include but are not limited to: Mango and Pepper, Mango and Broccoli and Pepper aptamers, etc.

In some embodiments, the two-color INFAD system can allow the detection of two nucleic acid templates, one of which can be an internal control for the INFAD method. Such two channel INFAD system can have enhanced reliability compared to one channel. In some embodiments, the internal control in the two-channel INFAD system can be used to distinguish a true negative from a false negative. This may allow the user to determine whether a failed reaction is a result of the inner reaction and/or outer reaction. Accordingly, INFAD primers may be modified to encode two colour aptamer sequences. Further by the addition of the two fluorophores two channel imaging is possible. Similar approaches can be used for three or more channel imaging, using three or more colour aptamer sequences.

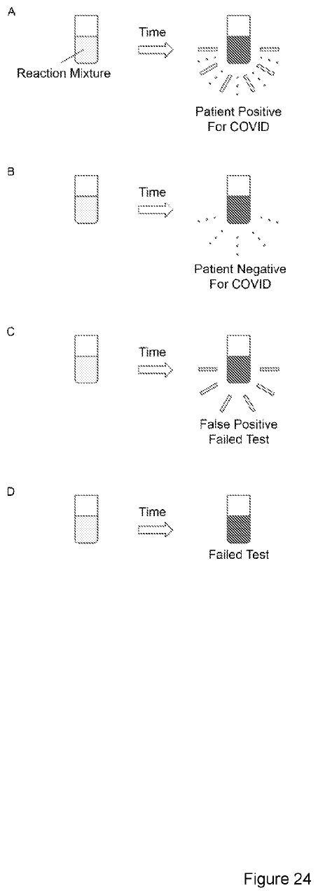

In some embodiments, two simultaneous isothermal reactions (e.g., NASBA reactions) can be performed in the same tube, as in FIG. 24, which shows a schematic of the possible outcomes of an internally controlled two colour assay. One reaction will have oligos that target RNA of interest (such as SARS-CoV-2) producing an aptamer with fluorescence (such as green fluorescence, represented by bar lines in FIG. 24). The second reaction will have oligos that target an internal control RNA (such as Human 18S ribosomal RNA) producing an aptamer with fluorescence (such as red fluorescence but not the same fluorescence as the first aptamer, represented by dashed lines in FIG. 24). The possible outcomes from reactions are: A. SARS-CoV-2 RNA is detected (bar lines, FIG. 24A) and Human ribosomal RNA detected (dashed lines, FIG. 24A); B. SARS-CoV-2 RNA is not detected (lack of bar lines, b) and Human ribosomal RNA detected (dashed lines, FIG. 24B); C. SARS-CoV-2 RNA detected but internal control Human ribosomal RNA was not detected, implicating either a false positive, or simply a failed test (FIG. 24C); Failed assay, unable to determine whether SARS-CoV-2 RNA is present, as internal control has failed (FIG. 24 D). RNA described above is only an example, any RNA can be replaced above with desired RNA. It is to be understood that this approach may be extended to three or more simultaneous isothermal reactions (e.g., NASBA reactions).

In some embodiments, Mango aptamers can be inserted into NASBA DNA primers to monitor the exponential synthesis of RNA reporter in an isothermal method. NASBA uses two primers, with the first serving as the initial reverse transcription primer and including a T7 promoter. After production of cDNA, the RNA in the newly formed heteroduplex can be degraded by RNase H allowing a second DNA primer to bind and be extended again by reverse transcriptase (RD. This produces a double stranded DNA template that can be transcribed by T7 RNA polymerase. As the resulting RNA can be utilized by RT, exponential amplification occurs (FIG. 1A). By modifying the second or bottom strand NASBA primer to code for a fluorogenic Mango aptamer, exponential RNA growth can be directly monitored by fluorescence (FIG. 1 B). The alteration of a DNA primer can dramatically reduce the complexity of NASBA and allows real-time monitoring. When combined with a nesting approach (FIG. 1C), this method can detect as little as 1.5 RNA molecules per μl of reaction.

In some embodiments, the present disclosure provides a composition that can be used in the methods as described herein. The composition can include fluorogenic aptamers conjugated to the oligonucleotide primers (for example, one or more of the nucleic acid molecules or compositions, as described herein) for isothermal amplification as described herein and their corresponding ligands, for example, dyes.

In some embodiments, the present disclosure provides a kit that can be used in the methods as described herein. In some embodiments, the kit may include one or more of the nucleic acid molecules or compositions, as described herein, together with instructions for amplification of a target nucleic acid sequence. In some embodiments, the amplification may be an isothermal amplification, such as nucleic acid sequence based amplification, Rolling Circle Amplification, Loop mediated isothermal amplification, Helicase dependent amplification, or Strand Displacement Amplification.

In some embodiments, the kit can include fluorogenic aptamers conjugated to the oligonucleotide primers (for example, one or more of the nucleic acid molecules or compositions, as described herein) for isothermal amplification as described herein and their corresponding ligands, for example, dyes. In some embodiments, the kit can be used to amplify the nucleic acid target sequence to an extent that permits the detection of the target sequence in the sample. In some embodiments, the kit can include instructions for use, or for performing the methods as described herein.

In some embodiments, the compositions, kits and methods as described herein can be used for example in the detection of extremely low concentrations of RNA and/or DNA templates, as well as high concentrations, in the field or laboratory for applications including but not limited to specific target gene detection and quantification, pathogen detection in clinically or scientifically relevant samples such as those from tissue culture, serum and plasma, disease marker detection in clinical samples, contaminant detection in environmental and controlled tissue culture samples, in vitro samples, in vivo imaging and localization, etc.

In some embodiments, the compositions, kits and methods as described herein can be used in food safety and food biosecurity applications, such as screening food products and materials used in food processing or packaging for the presence of pathogens in biological and/or non-biological samples. In other embodiments, the methods provided herein can be used for anti-counterfeit applications, such as confirming that pharmaceuticals are genuine or confirming the identity of high value items that have been fabricated or are known to contain specific nucleic acid species.

In some embodiments, the compositions, kits and methods as described herein can be used in conjunction with point of care devices.

The present invention will be further illustrated in the following examples.

### EXAMPLES

### Example 1

Material and Methods

Target RNA Generation

Colony PCR reactions were performed using the respective PCR primers shown in Table 1 using 5 pM plasmid template, Taq (NEB, 10 U), 0.2 mM each dNTP, 10 mM TRIS buffer pH 8.3, 50 mM KCl, 1.5 mM MgCl₂, and 0.01% gelatin, followed by cloning into a pGEM-T Easy Vector (Promega). Sequences were confirmed by Eurofins tube sequencing. Using plasmid as template, PCR reactions were carried out followed by ethanol precipitation in 300 mM NaCl and 70% Ethanol. Pellets were suspended one-tenth the PCR reaction volume for a 10× stock. Transcriptions were carried out using 2× template, T7 RNA polymerase (ABM), in 8 mM GTP, 5 mM CTP and ATP, 2 mM UTP, 40 mM TRIS buffer pH 7.9, 2.5 mM spermidine, 26 mM MgCl₂, and 0.01% Triton X-100. RNA was purified via 5% PAGE (19:1 Acrylamide:bis), rotation overnight in 300 mM NaCl and ethanol precipitation. Concentrations were determined using a SHIMADZU dual beam spectrophotometer.

TABLE 1
Sequences of primers and targets

| Identifier | Sequence |
|---|---|
| CIpB Short Target E. coli | GGA CGU CUG GAA GAA CGU GGU UAU GAA AUC CAC |
| | AUU UCU GAC GAG GCG CUG AAA CUG CUG AGC GAG |
| | AAC GGU UAC GAU CCG GUC UAU GGU GCA CGU CCU |
| | CUG AAA CGU GCA AUU CAG CAG CAG AUC GAA AAC |
| | CCG CUG GCA CAG CAA AUA CUG UCU GGU GAA UUG |
| | GUU CCG GGU AAA GUG AUU CGC CUG GAA GUU AAU |
| | GAA GAC CGG AUU GUC GCC GUC CAG UAA AUG AUA |
| | AAA CGA GCC CUU CGG G (SEQ ID NO: 3) |
| Ec PCR Forward Primer | CTT TAA TAC GAC TCA CTA TAG GAC GTC TGG AAG |
| | AAC GTG GTT ATG (SEQ ID NO: 5) |
| Ec PCR Reverse Primer | CCC GAA GGG CTC GTT TTA TCA TTT A (SEQ ID NO: 6) |
| P1: Ec T7 Outer NASBA | AAT TCT AAT ACG ACT CAC TAT AGG GAG AAG GCT |
| Top Primer | GGA CGG CGA CAA TCC GGT CTT CA (SEQ ID NO: 7) |
| P2A: Ec Mango III A1OU | GGC ACG TAC GAA TAT ACC ACA TAC CAA ACC TTC |
| Outer NASBA Bottom | CTT CGT ACG TGC CAA ATC CAC ATT TCT GAC GAG G |
| Primer | (SEQ ID NO: 8) |
| P2B: Ec Outer NASBA | AAA TCC ACA TTT CTG ACG AGG (SEQ ID NO: 9) |
| Bottom Primer (- Mango) | |
| P2C: Ec Mango III Outer | GGC ACG TAC GAA TAT ACC ACA TAC CAA TCC TTC |
| NASBA Bottom Primer | CTT CGT ACG TGC CAA ATC CAC ATT TCT GAC GAG G |
| | (SEQ ID NO: 10) |
| P3: Ec Inner T7 NASBA | AAT TCT AAT ACG ACT CAC TAT AGG GAA GGA AGT |
| Top Primer | CTG GTG AAT TGG TTC CGG (SEQ ID NO: 11) |
| P4: Ec Inner Mango III | GGC ACG TAC GAA TAT ACC ACA TAC CAA ACC TTC |
| A1OU NASBA Bottom | CTT CGT ACG TGC CTT CCA GGC GAA TCA CTT TAC |
| Primer | (SEQ ID NO: 12) |
| CIpB Short Target P. | GGU CGC CUG GCC GAG CGU GAG CUU GAC CUG GAG |
| fluorescens | CUG AGC AGC GAG GCG UUG GAC AAG CUG AUU GCG |
| | GUC GGU UAC GAC CCG GUG UAU GGC GCA CGG CCA |
| | CUU AAA CGU GCG AUC CAG CGC UGG AUC GAA AAC |
| | CCA CUG GCA CAG UUG AUC CUG UCG GGC AGC UUC |
| | AUG CCA GGC ACC CGC GUG ACG GCC ACG GUG GAA |
| | AAC GAC GAA AUC GUC UUC CAC UAA GCC CAG CCU |
| | GUA GGG UUA UUA GAG A (SEQ ID NO: 4) |
| Pf PCR Forward Primer | CTT TAA TAC GAC TCA CTA TAG GTC GCC TGG CCG |
| | AGC GTG AGC TTG (SEQ ID NO: 13) |
| Pf PCR Reverse Primer | TCT CTA ATA ACC CTA CAG GCT GGG C (SEQ ID NO: 14) |
| P5: Pf T7 Outer NASBA | CTT TAA TAC GAC TCA CTA TAG GGA GGC TGG GCT |
| Top Primer | TAG TGG AAG A (SEQ ID NO: 15) |
| P6: Pf Outer NASBA | GAG CAG CGA GGC GTT GGA CA (SEQ ID NO: 16) |
| Bottom Primer | |
| P7: Pf Inner T7 NASBA | CTT TAA TAC GAC TCA CTA TAG GGC GAA AAC CCA |
| Top Primer | CTG GCA CAG T (SEQ ID NO: 17) |
| P8: Pf Inner Mango III | GGC ACG TAC GAA TAT ACC ACA TAC CAA ACC TTC |
| A1OU NASBA Bottom | CTT CGT ACG TGC CCG CGG GTG CCT GGC ATG AAG |
| Primer | (SEQ ID NO: 18) |
| Linear RCA template | AAG TTT TCA GCT GCT TGC CCT ATA GTG AGT |
| | CGT ATT AGG CAC GTA CGA ATA TAC CAC ATA |
| | CCA ATC CTT CCT TCG TAC GTG CCC GGA AAA |
| | GTT TGA AGA G (SEQ ID NO: 19) |
| Target RCA splint | AAA AGC GGA AAA GTT TGA AGA GAA GTT TTC |
| | AGC TGC TTG CGC TTA TCC TAT AGT GAG TCG |
| | TAT TA (SEQ ID NO: 20) |
| T7 promoter top strand | CTT TAA TAC GAC TCA CTA TAG G (SEQ ID NO: 21) |
| sequence | |

Mango-NASBA

NASBA primers were chosen for RNA amplification using a short segment of E. coli or P. fluorescens CIpB mRNA as detecting template (shown as “CIpB Short Target E. coil” and “CIpB Short Target P. fluorescens” respectively, Table 1). Reactions were carried out using NASBA buffer mix (Life Sciences, NECB-1-24), nucleotide mix (Life Sciences, NECN-1-24), 250 nM of each primer (IDT), T7 containing cDNA primer P1 and Mango template containing reverse primer “P2A adapted from Heijnen and Medema (2009), 480 nM TO1-Biotin (ABM), and NASBA enzyme mix (Life Sciences, NEC-1-24). NASBA reactions were mixed excluding the enzyme mix and RNA target was added to a final of either 0, 25 aM, 25 fM, 25 pM. RNA was heated to 65° C. for 2 min and brought down to 41° C. for 5 minutes in a MJ research PTC-100 thermocycler. To begin the reaction, enzyme mix was added the reactions and they were incubated at 41° C. in 8-tube strips with optical caps (Applied Biosystems, catalog #4358293, 4323032) on a StepOne Real-Time PCR System (Applied Biosystems) Set to read SYBR Green reagents in the following program: 1. Ramp to 41° C., read, 2. Hold 41° C. for 30 seconds, read, 3. Repeat step 2 until 480 cycles complete. Experiments of supplementary figure S6 were carried out with a P2A that did not carry the A10U mutation (WT Mango III was used).

Nested Mango-NASBA

Outer amplification reactions were done as described above, however P2A was replaced with P2B which lacks the Mango template. Reactions were stopped at 40 minutes by the addition of 5 μL EDTA to a final concentration of 10 mM in a final volume of 25 μL and flash frozen in either liquid nitrogen or ethanol cooled with. Aliquots from these reactions were diluted one hundred-fold into the inner nested Mango-NASBA reactions (20 μL) prepared as above except using T7 promoter containing cDNA primer P3 and Mango template containing reverse primer P4. Reactions were monitored for fluorescence of TO1-Biotin in real time again using the instrument above.

Detection of E. coli in the Presence of Conditioned Mammalian Cell Culture Media

LB media was inoculated with E. coli and concentration was monitored by absorbance at 600 nm (cell number calculated using Agilent online tool). An aliquot of 10⁸ cells treated to heat shock at 41° C. for 10 min to induce CIpB RNA in the cells before being pelleted at 4000 g for 4 min. The cells were resuspended in 50 μL of depleted cell culture media (MCF7, media that is thrown out during passaging of cells) and incubated at 41° C. for 3 min. Samples were pelleted at 4000 g for 4 min before subject to a Nucleospin RNA kit (Macherey-Nagel) using recommended protocol with the exceptions of avoiding the DNase step and elution was performed using 2 mM EDTA. Total nucleic acid samples were used for nested Mango-NASBA reactions as described above. A negative control sample for nested Mango NASBA was an extraction of nucleic acid from depleted media containing no E. coli cells treated to the same extraction procedure.

PAGE Mango Visualization

Samples to be visualized on PAGE were added to 3 volumes formamide with 20 mM EDTA added and heated to 90° C. for 5 minutes. Samples were loaded and run via 8% PAGE (19:1 Acrylamide:bis). Post staining of the gels was performed in 100 mL of 1× WB (140 mM KCl, 1 mM MgCl2, 10 mM NaH₂PO₄ pH 7.2, 0.05% Tween-20) including 20 nM TO1-Biotin, and Mango-NASBA bands were imaged on a GE A1600RGB imager as previously described³⁴). Alternatively, gels were stained with 1× SYBR Safe under the same conditions.

Sequence Alignment

Sequences were aligned using Geneious software and aligning using the Clustal method.

Results

Sensitivity of Fluorogenic Aptamer-NASBA

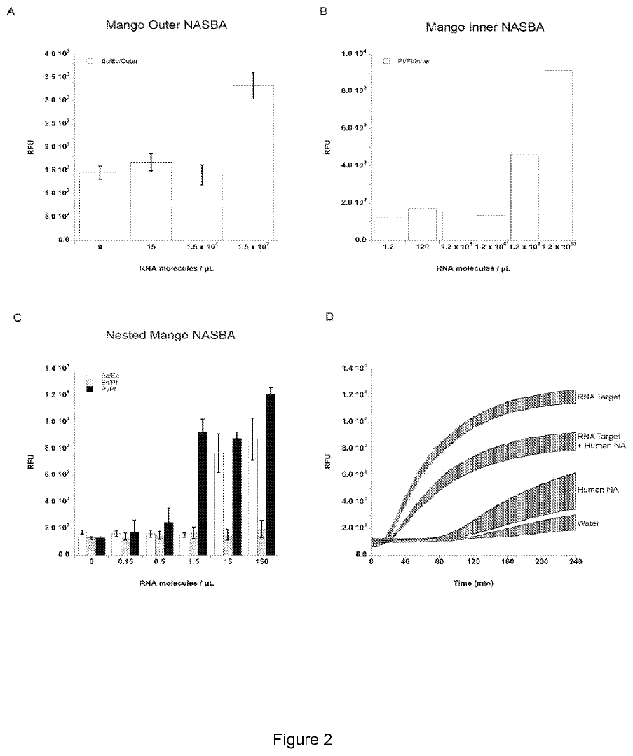

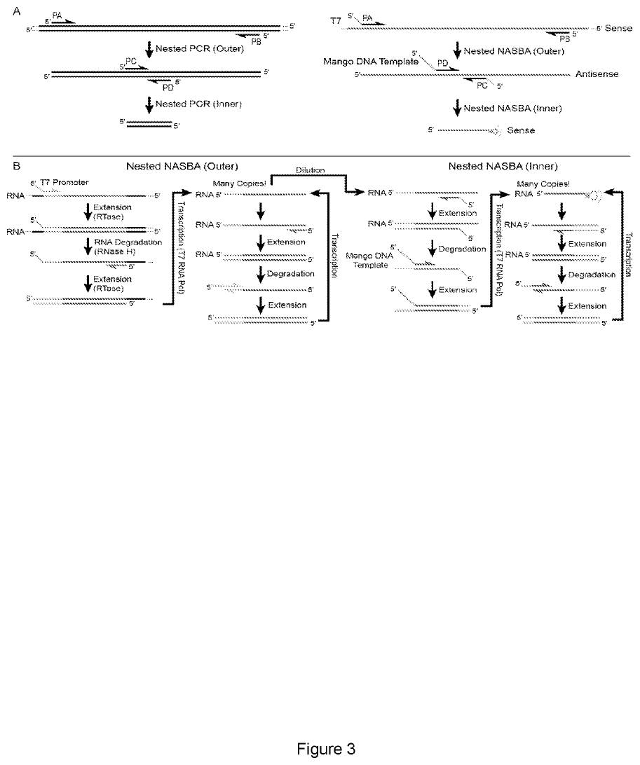

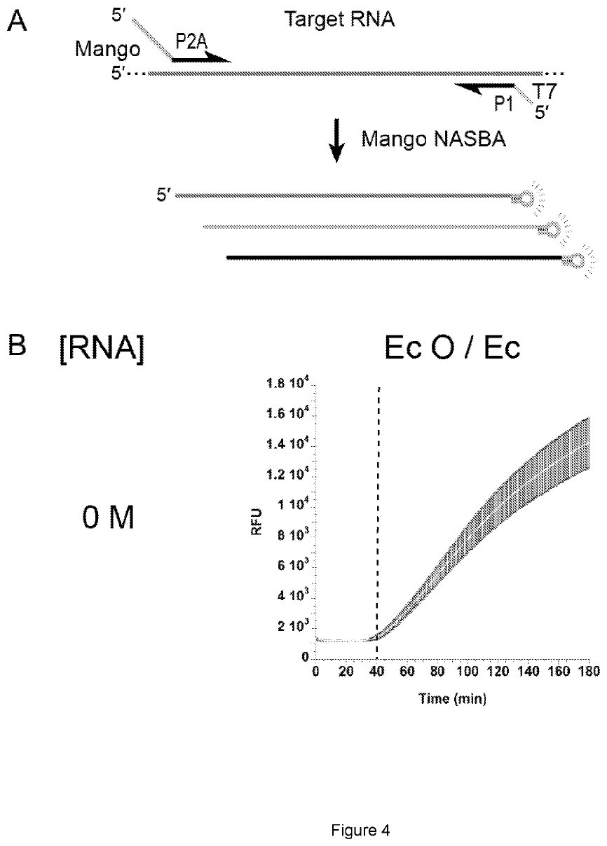

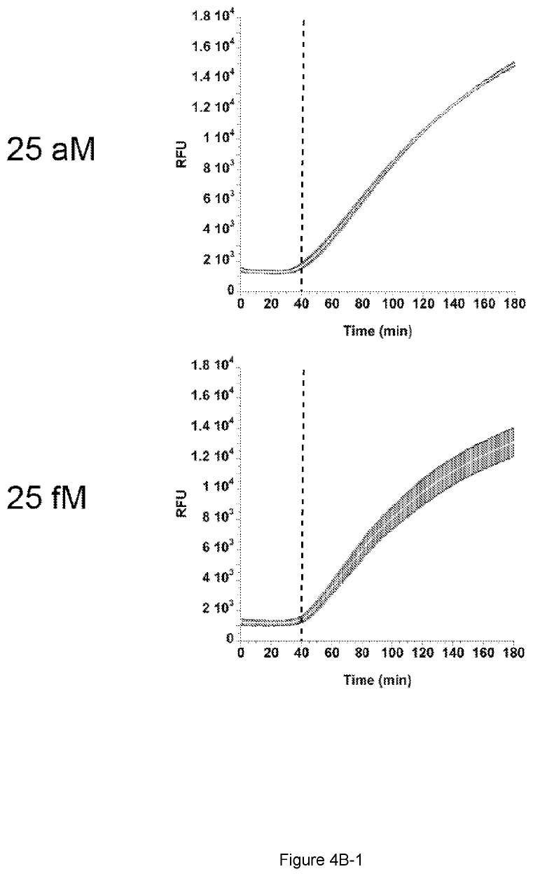

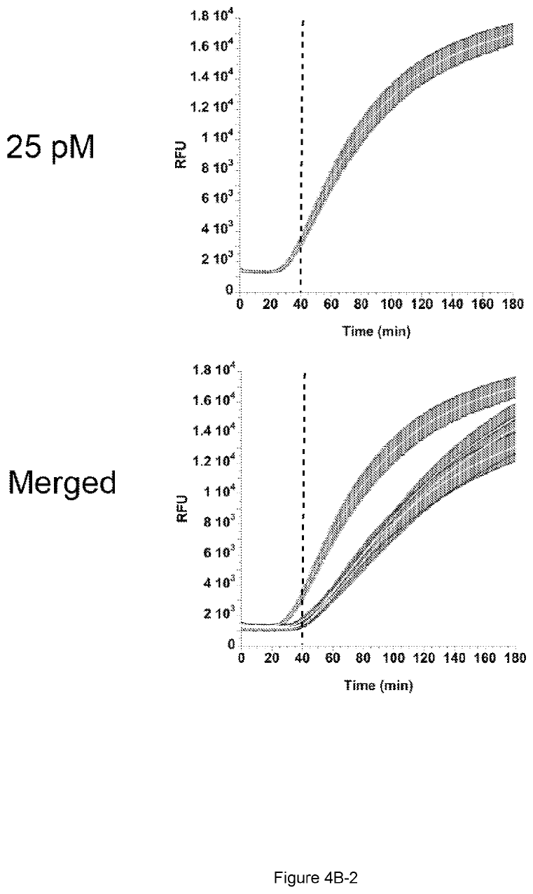

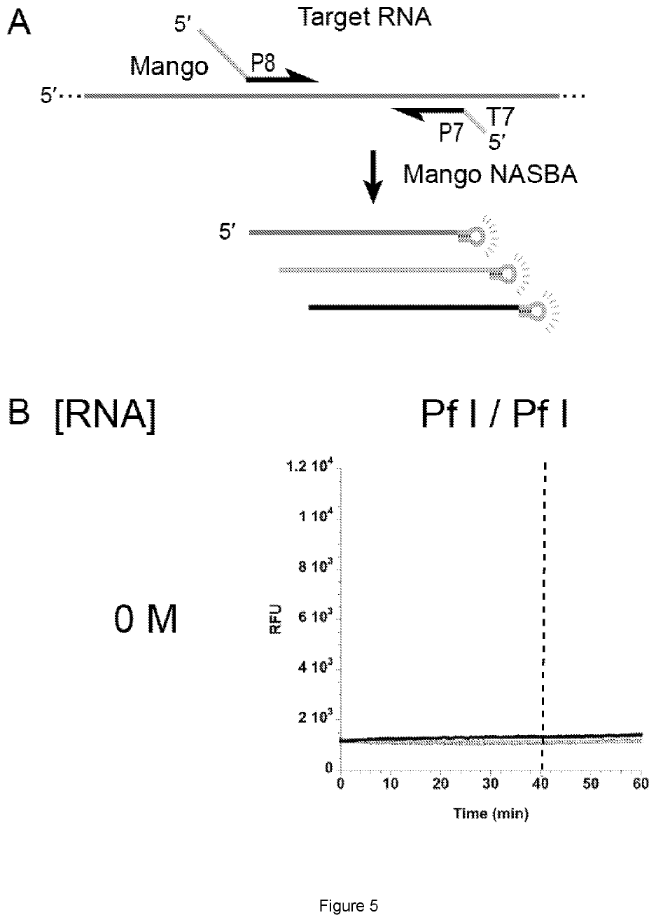

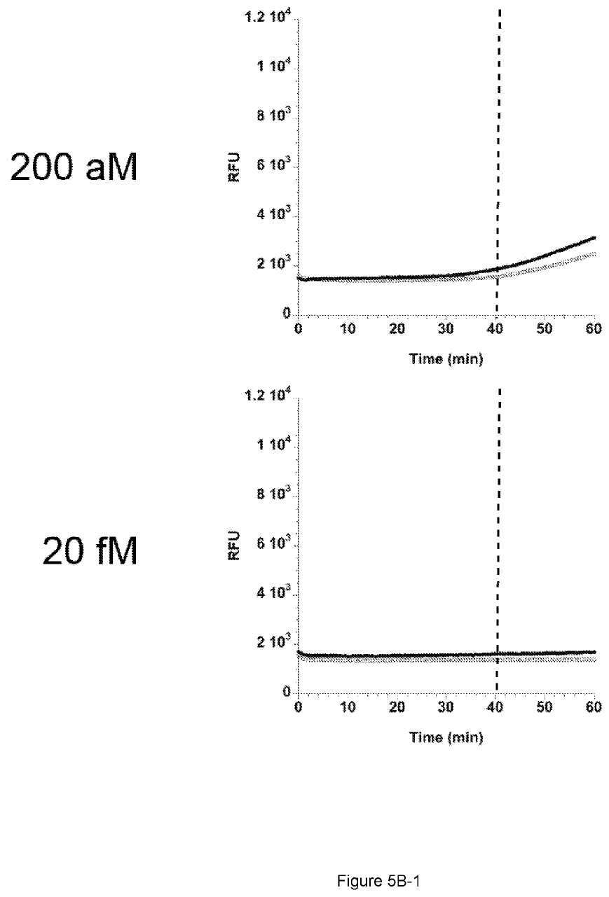

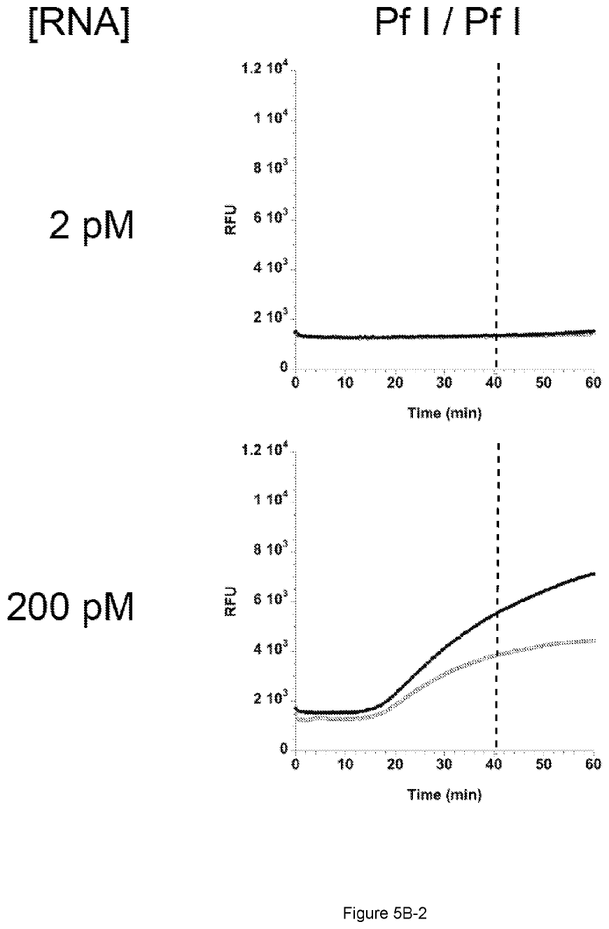

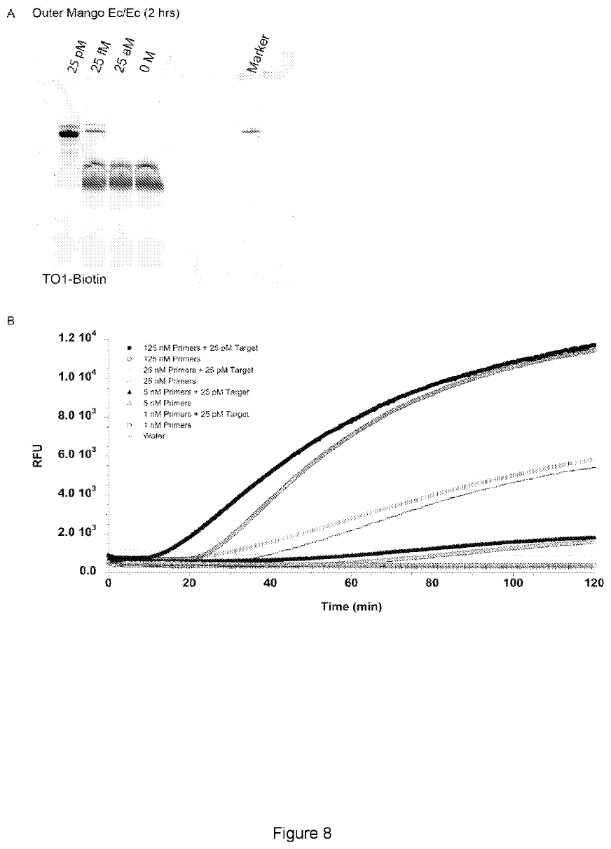

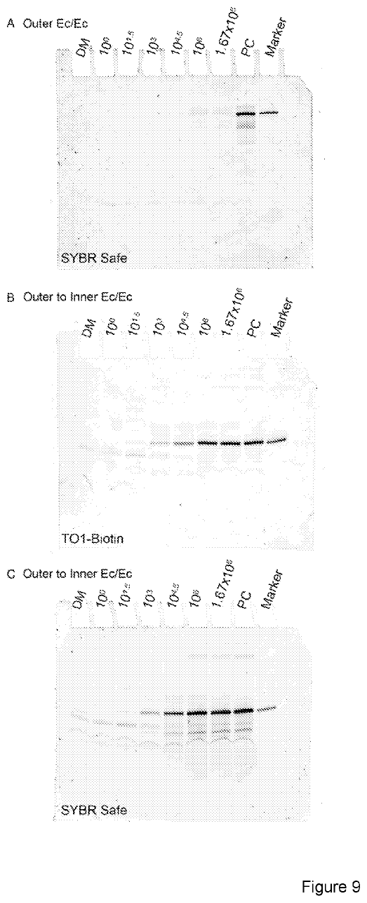

Using commercially available NASBA enzyme mix we could detect as little as ˜25 pM (15 000 000 RNA/μL of reaction, FIGS. 2A, 4, 5, Primers P1/P2A) of E. coli CIpB RNA template over a background signal that amplified rapidly even in the complete absence of RNA template (FIGS. 4, 8) using Mango NASBA. This intrinsic level of sensitivity was not primer or template specific, as P. fluorescens CIpB RNA template could be detected with similar sensitivity using primers that hybridized much closer together (FIG. 2B, Primers P7/P8).

Sensitivity of Nested Fluorogenic Aptamer-NASBA

Outer primers were identical in sequence to those used in Mango NASBA, but P2A now lacked the Mango III tag (P1/P2B, Table 1).

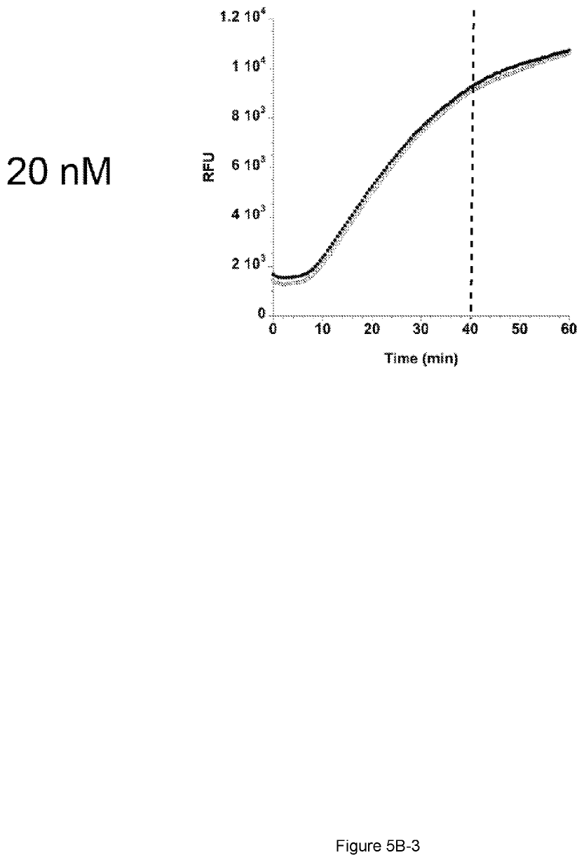

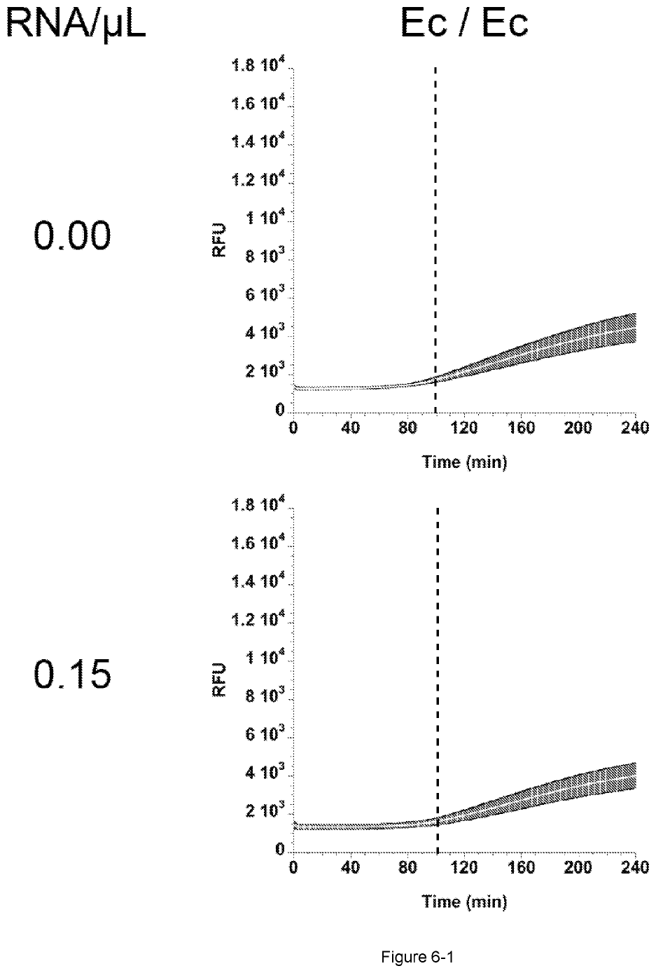

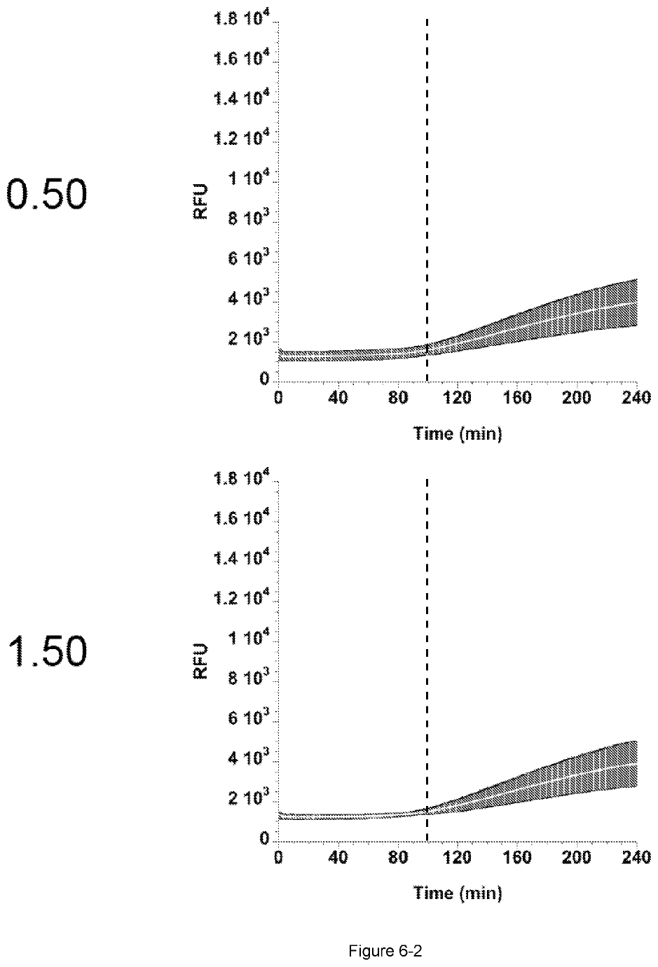

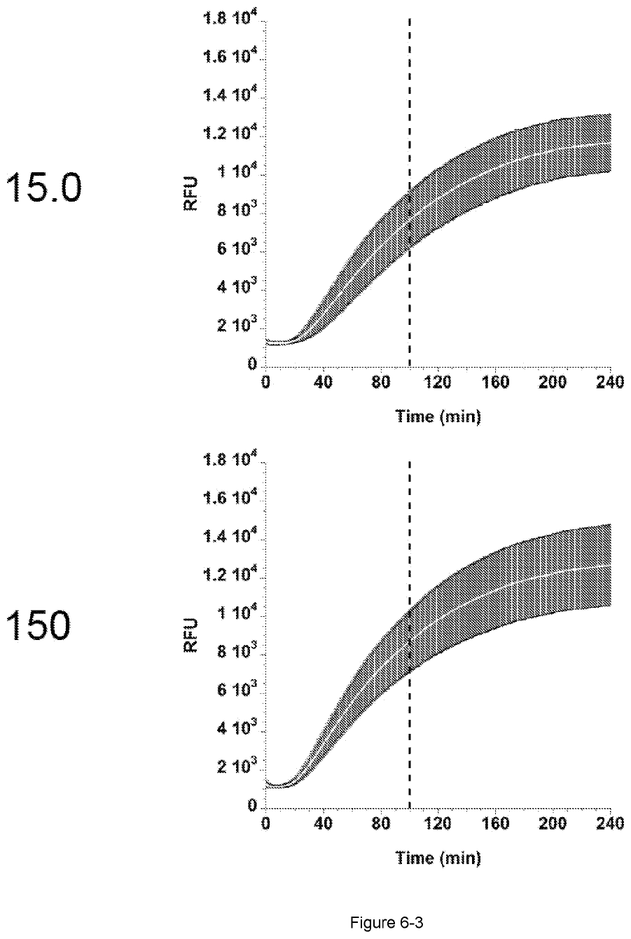

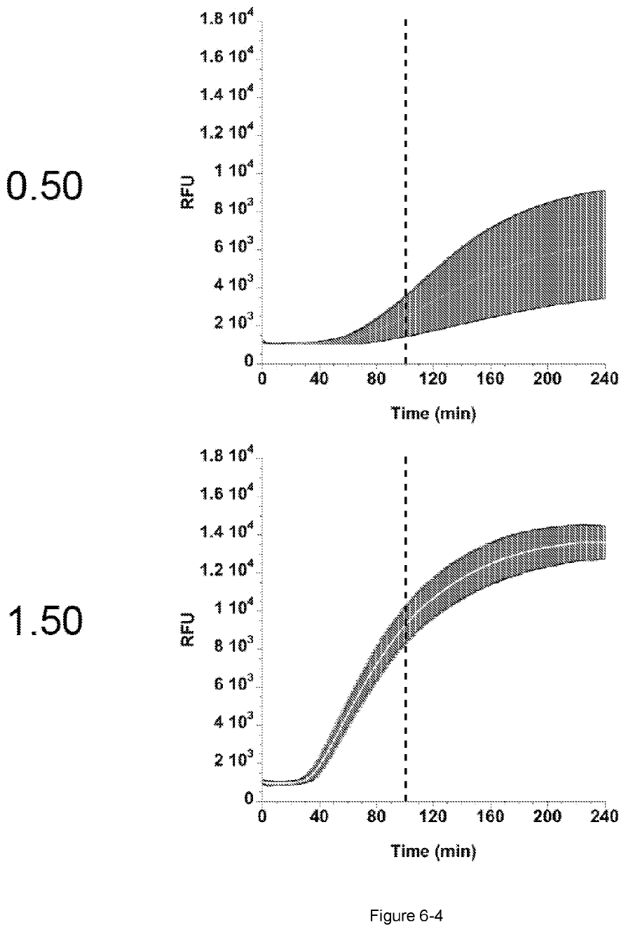

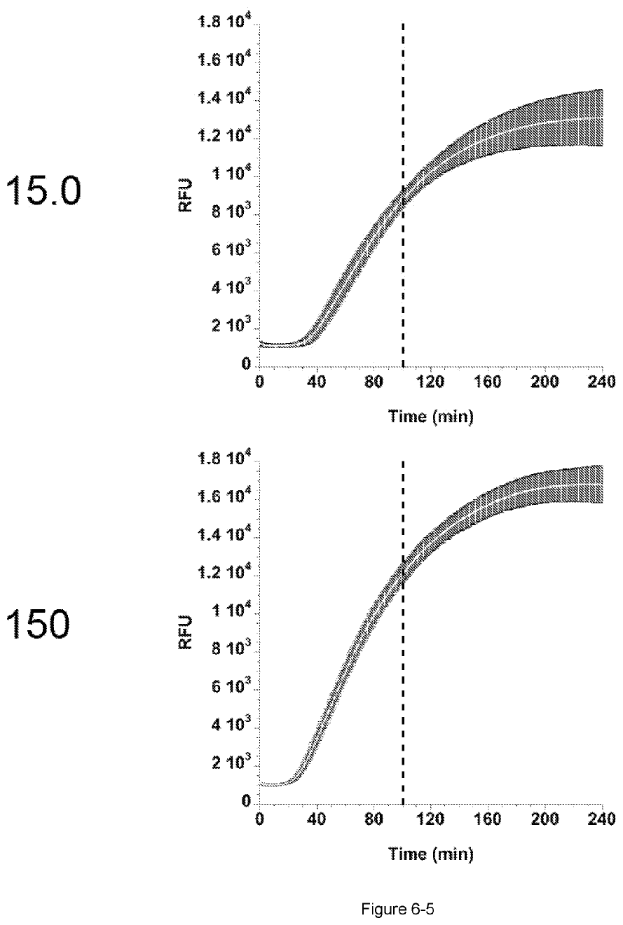

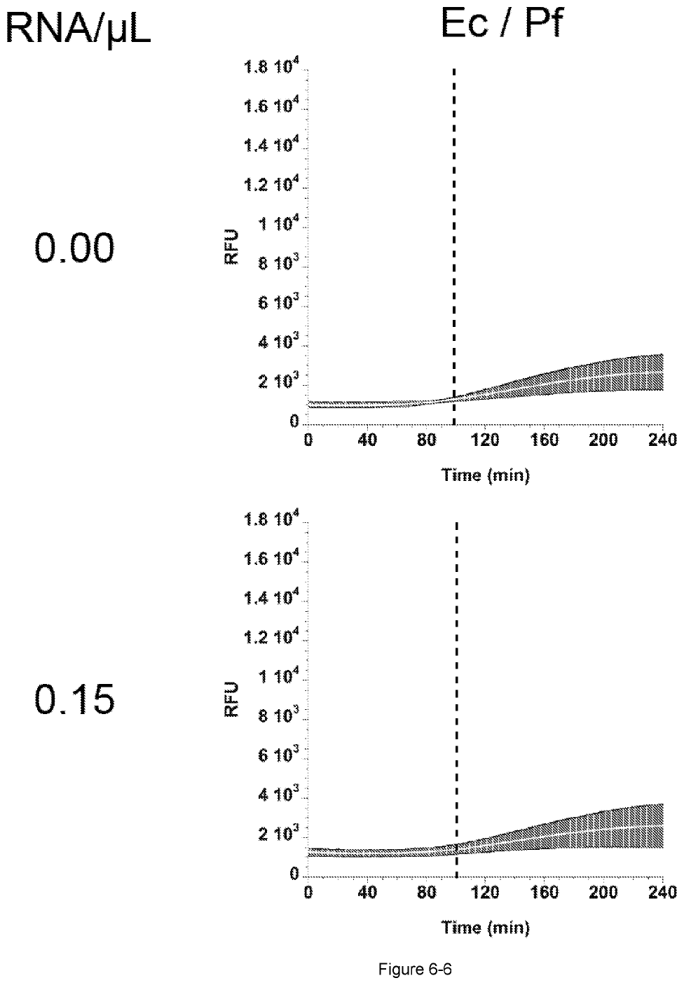

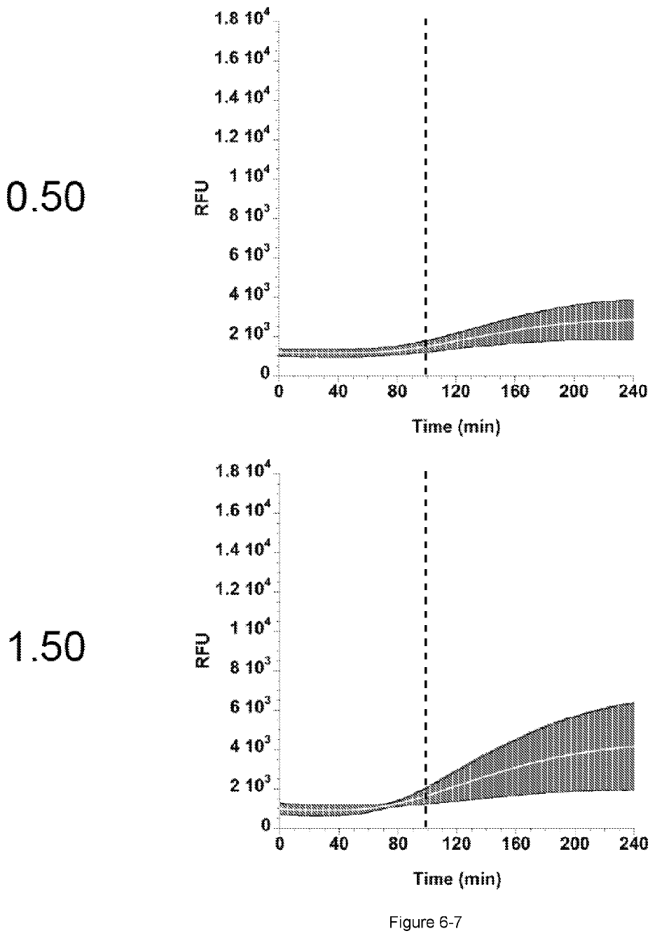

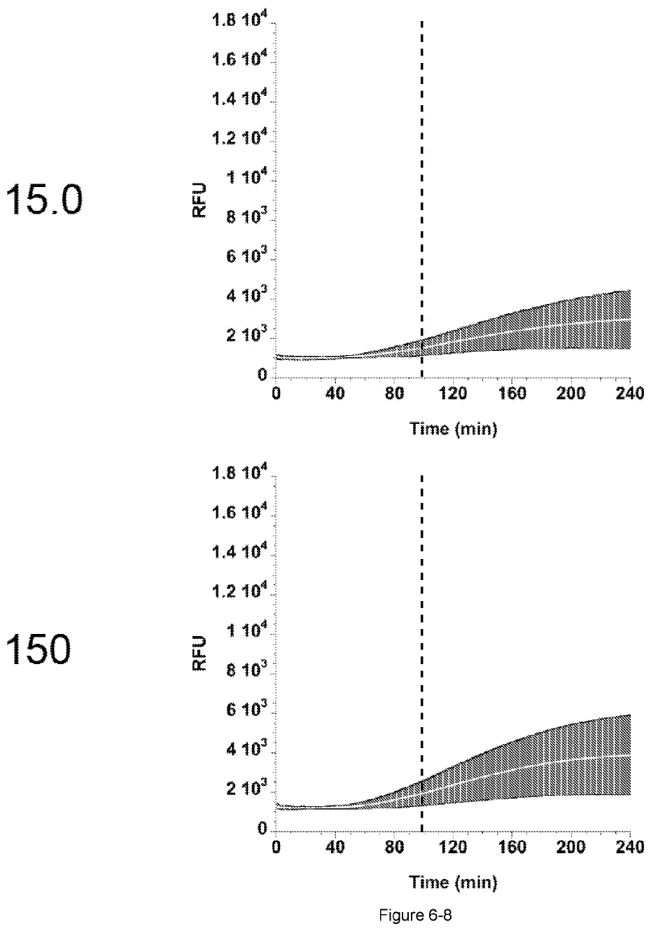

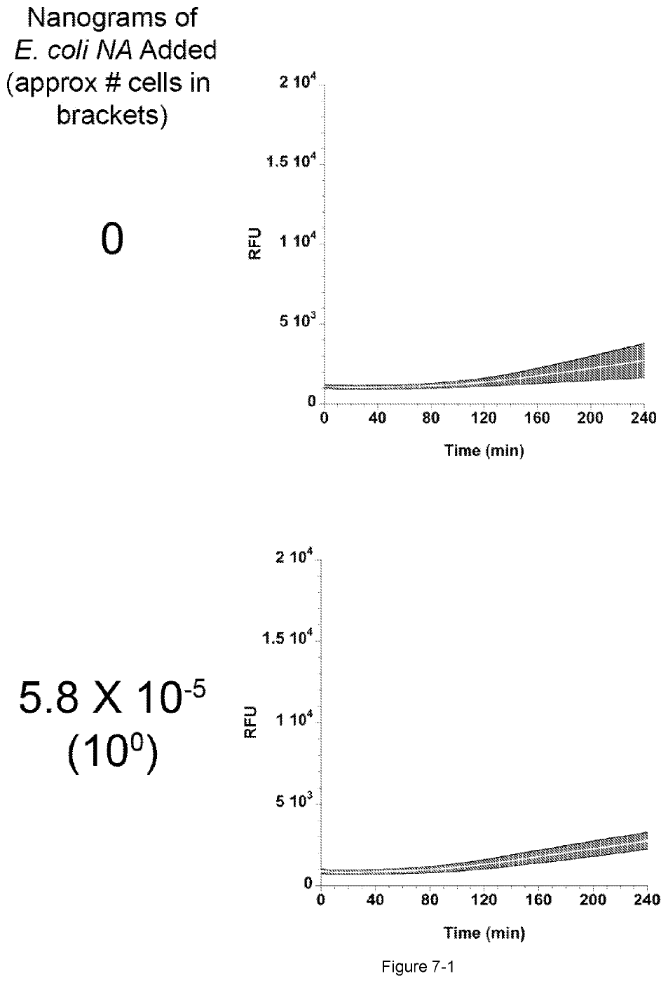

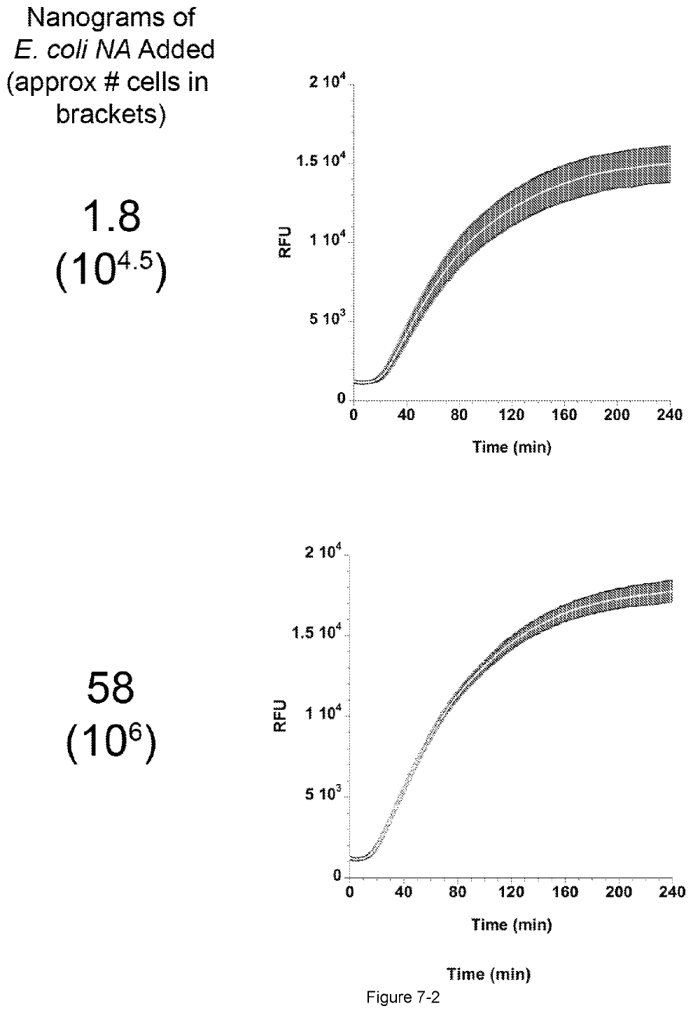

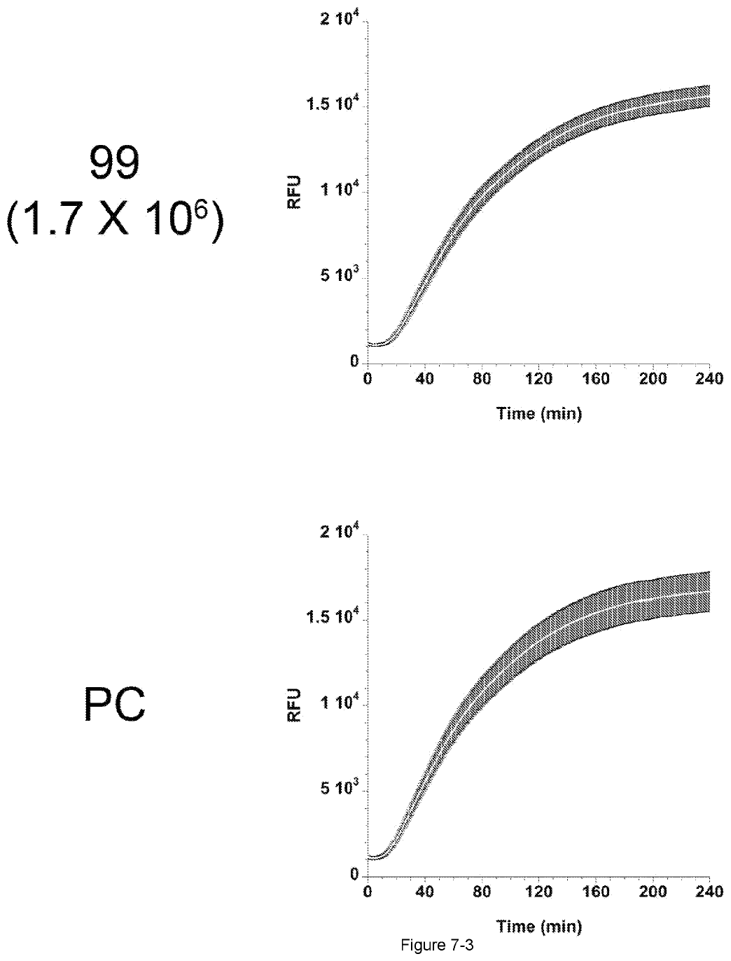

At the 0.25 μM concentration of primers used in the outer NASBA reaction, we found that dilution by 100-fold was sufficient to suppress NASBA activity (FIG. 8B). After dilution by 100-fold into a fresh inner NASBA reaction after a 40 min incubation of this outer NASBA reaction and using inner NASBA Mango primers (P3/P4), we could now easily detect 15 RNA/μL of E. coli CIpB template sequence (Ec/Ec reactions, FIGS. 2C, 6). Using the same dilution strategy, we tested Nested Mango NASBA on the P. fluorescens template and could detect 1.5 RNA molecules/μL using P. fluorescens-specific nested Mango NASBA primers (Outer: P5/P6, Inner: P7/P8. FIGS. 2C, 6). This approach improved sensitivity by 6 orders of magnitude when using the same E. coli target RNA.

Nested Fluorogenic Aptamer NASBA Specificity and Robustness

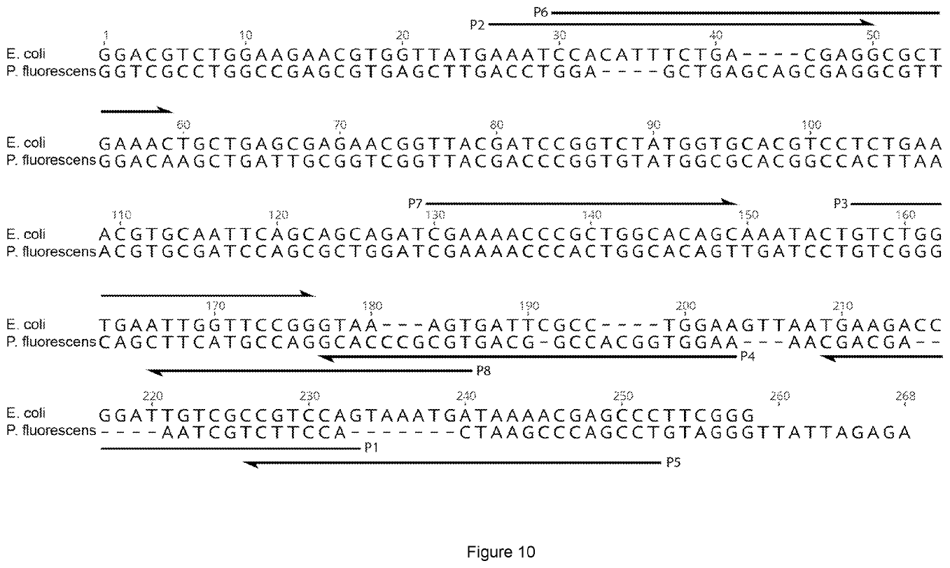

E. coli and P. Fluorescens CIpB template differs by 78 nt in the amplified region of which 65 nt are in primer hybridization regions (alignment FIG. 10). When primers designed to target E. coli were used with a P. fluorescens target, fluorescence remains within error the same as the 0 RNA/μL reaction control (Ec/Pf, FIGS. 2C, 6). To see if nested Mango NASBA reactions remain viable in a large background of human nucleic acid, E. coli CIpB target was mixed with or without a very large excess of human total nucleic acid (150 RNA molecules/μL final CIpB Short Target E. coli, 5 ng/μL human total nucleic acid) and Nested RNA Mango performed using the Ec primers (Outer: P1/P2B, Inner: P3/P4, FIG. 2D). While the emergence of time dependent signal was slightly decreased, a robust signal was still observed in these conditions suggesting that Nested Mango NASBA is largely robust to nucleic acid amplification artifacts despite the addition of such a large amount of human RNA and DNA.

### Example 2

RT-PCR Primers were designed to amplify 1 kb fragments from cultured SARS-CoV-2 (COVID-19; Table 2).

TABLE 2
SARS-CoV-2 Target (TN, N = 2 to 6) Generation Primers. Each primer set
generates a 1003 nt long positive strand viral fragment.

| Identifier (NCBI:NC_045512.2 loci) | Sequence |
|---|---|
| SARS-CoV-2 T2: PCR Forward Primer | CTT TAA TAC GAC TCA CTA TAG GGG TGC |
| Start 2762 | AAG GTT ACA AGA GTG TGA ATA TC (SEQ |
| | ID NO: 22) |
| SARS-CoV-2 T2: PCR Reverse Primer | ACA CAA ACT CTT AAA GAA TGT ATA GGG |
| End 3762 | TCA (SEQ ID NO: 23) |
| SARS-CoV-2 T2: cDNA Primer | GTA GAC ATT TGT GCG AAC AG (SEQ ID |
| 3768 - 3787 | NO: 24) |
| SARS-CoV-2 T3: PCR Forward Primer | CTT TAA TAC GAC TCA CTA TAG GGT GTA |
| Start 3376 | TAC ATT AAA AAT GCA GAC ATT GTG GAA |
| | G (SEQ ID NO: 25) |
| SARS-CoV-2 T3: PCR Reverse Primer | CAG TTC CAA GAA TTT CTT GCT TCT CAT |
| End 4376 | TA (SEQ ID NO: 26) |
| SARS-CoV-2 T3: cDNA Primer | AGC ATT TCT CGC AAA TTC CA (SEQ ID NO: |
| 4382 - 4401 | 27) |
| SARS-CoV-2 T4: PCR Forward Primer | CTT TAA TAC GAC TCA CTA TAG GGA TCT |
| Start 21194 | TTA TAA GCT CAT GGG ACA CTT CG (SEQ |
| | ID NO: 28) |
| SARS-CoV-2 T4: PCR Reverse Primer | TTA ATA GGC GTG TGC TTA GAA TAT ATT |
| End 22194 | TTA AAA TAA C (SEQ ID NO: 29) |
| SARS-CoV-2 T4: cDNA Primer | ACC CTG AGG GAG ATC ACG CA (SEQ ID |
| 22200 - 22219 | NO: 30) |
| SARS-CoV-2 T5: PCR Forward Primer | CTT TAA TAC GAC TCA CTA TAG GGT GCC |
| Start 21585 | ACT AGT CTC TAG TCA GTG TG (SEQ ID NO: |
| | 31) |
| SARS-CoV-2 T5: PCR Reverse Primer | AAC TTC ACC AAA AGG GCA CAA GTT TG |
| End 22585 | (SEQ ID NO: 32) |
| SARS-CoV-2 T5: cDNA Primer | CAG ATG CAA ATC TGG TGG CG (SEQ ID |
| 22591 - 22610 | NO: 33) |
| SARS-CoV-2 T6: PCR Forward Primer | CTT TAA TAC GAC TCA CTA TAG GGA AGA |
| Start 27744 | AAG ACA GAA TGA TTG AAC TTT CAT TAA |
| | TTG AC (SEQ ID NO: 34) |
| SARS-CoV-2 T6: PCR Reverse Primer | GAT TGC AGC ATT GTT AGC AGG ATT G |
| End 28744 | (SEQ ID NO: 35) |
| SARS-CoV-2 T6: cDNA Primer | TTG TTC CTT GAG GAA GTT GT (SEQ ID NO: |
| 28750 - 28769 | 36) |

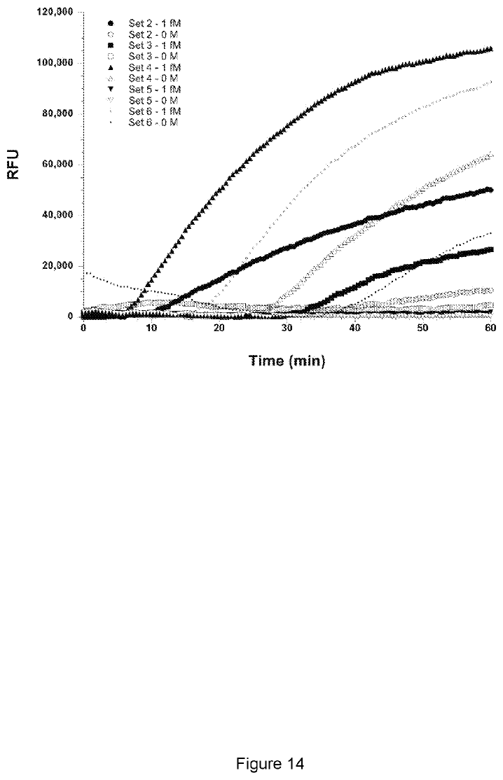

Following transcription, the resulting RNA (1 fM) was subjected to nested Mango NASBA using the NASBA Life Sciences (LS) liquid NASBA kit. Five sets of primers were designed to amplify 100 nt regions centered within these regions. 4 out of 5 primers sets (see Table 3) were successfully able to amplify COVID-19 RNA, with set producing the fastest rise time and the highest fluorescent signal (FIG. 14).

TABLE 3
NASBA Primer Sets (SN N = 2 to 6). Each set designed against respective
numbered target

| Identifier | Sequence |
|---|---|
| SARS-CoV-2 Set 2 | CTT TAA TAC GAC TCA CTA TAG GGT TCC ATC |
| P1: T7 Outer NASBA Top Primer | TCT AAT TGA GGT T (SEQ ID NO: 37) |
| SARS-CoV-2 Set 2 | TAG TCA ACA AAC TGT TGG TC (SEQ ID NO: 38) |
| P2: Outer Bottom Primer | |
| SARS-CoV-2 Set 2 | CTT TAA TAC GAC TCA CTA TAG GGG CAG TGA |
| P3: T7 Inner NASBA Top Primer | GGA CAA TCA GAC A (SEQ ID NO: 39) |
| SARS-CoV-2 Set 2 | GGC ACG TAC GAA TAT ACC ACA TAC CAA ACC |
| P4: Inner Mango III A1OU Bottom | TTC CTT CGT ACG TGC CAC AAT TGT TTG AAT |
| NASBA Primer | AGT AGT (SEQ ID NO: 40) |
| SARS-CoV-2 Set 3 | CTT TAA TAC GAC TCA CTA TAG GGC TTT CAG |
| P1: T7 Outer NASBA Top Primer | TTA TAA ATG GCT T (SEQ ID NO: 41) |
| SARS-CoV-2 Set 3 | AGC TTT TTG GAA ATG AAG AG (SEQ ID NO: 42) |
| P2: Outer Bottom Primer | |
| SARS-CoV-2 Set 3 | CTT TAA TAC GAC TCA CTA TAG GGG CAA GTT |
| P3: T7 Inner NASBA Top Primer | GAA CAA AAG ATC G (SEQ ID NO: 43) |
| SARS-CoV-2 Set 3 | GGC ACG TAC GAA TAT ACC ACA TAC CAA ACC |
| P4: Inner Mango III A1OU Bottom | TTC CTT CGT ACG TGC C TTC CTC TTT AGG AAT |
| NASBA Primer | CTC AG (SEQ ID NO: 44) |
| SARS-CoV-2 Set 4 | CTT TAA TAC GAC TCA CTA TAGGG GGA AAA |
| P1: T7 Outer NASBA Top Primer | GAA AGG TAA GAA CA (SEQ ID NO: 45) |
| SARS-CoV-2 Set 4 | TAC CCC CTG CAT ACA CTA AT (SEQ ID NO: 46) |
| P2: Outer Bottom Primer | |
| SARS-CoV-2 Set 4 | CTT TAA TAC GAC TCA CTA TAG GGT ACC CTG |
| P3: T7 Inner NASBA Top Primer | ACA AAG TTT TCA G (SEQ ID NO: 47) |
| SARS-CoV-2 Set 4 | GGC ACG TAC GAA TAT ACC ACA TAC CAA ACC |
| P4: Inner Mango III A1OU Bottom | TTC CTT CGT ACG TGC CTT GAA TGT AAA ACT |
| NASBA Primer | GAG GAT (SEQ ID NO: 48) |
| SARS-CoV-2 Set 4 | CTT TAA TAC GAC TCA CTA TAG GGT AAG AAC |
| P5: T7 Non-Nested NASBA Top | AAG TCC TGA GTT G (SEQ ID NO: 49) |
| Primer | |
| SARS-CoV-2 Set 4 | GGC ACG TAC GAA TAT ACC ACA TAC CAA ACC |
| P6: Non-Nested Mango III A1OU | TTC CTT CGT ACG TGC CCA GAT CCT CAG TTT |
| Bottom NASBA Primer | TAC ATT (SEQ ID NO: 50) |
| SARS-CoV-2 Set 5 | CTT TAA TAC GAC TCA CTA TAG GGT TCC CTA |
| P1: T7 Outer NASBA Top Primer | AGA TTT TTG AAA T (SEQ ID NO: 51) |
| SARS-CoV-2 Set 5 | AGT TTA TTC TAG TGC GAA TA (SEQ ID NO: 52) |
| P2: Outer Bottom Primer | |
| SARS-CoV-2 Set 5 | CTT TAA TAC GAC TCA CTA TAG GGT TTG AAT |
| P3: T7 Inner NASBA Top Primer | ATG TCT CTC AGC C (SEQ ID NO: 53) |
| SARS-CoV-2 Set 5 | GGC ACG TAC GAA TAT ACC ACA TAC CAA ACC |
| P4: Inner Mango III A1OU Bottom | TTC CTT CGT ACG TGC CTC CTT CAA GGT CCA |
| NASBA Primer | TAA GAA (SEQ ID NO: 54) |
| SARS-CoV-2 Set 6 | CTT TAA TAC GAC TCA CTA TAG GGT TTT AGT |
| P1: T7 Outer NASBA Top Primer | TTG TTC GTT TAG A (SEQ ID NO: 55) |
| SARS-CoV-2 Set 6 | GTT GTT CGT TCT ATG AAG AC (SEQ ID NO: 56) |
| P2: Outer Bottom Primer | |
| SARS-CoV-2 Set 6 | CTT TAA TAC GAC TCA CTA TAG GGT TTT TAG |
| P3: T7 Inner NASBA Top Primer | AGT ATC ATG ACG T (SEQ ID NO: 57) |
| SARS-CoV-2 Set 6 | GGC ACG TAC GAA TAT ACC ACA TAC CAA ACC |
| P4: Inner Mango III A1OU Bottom | TTC CTT CGT ACG TGC CTG AAA TCT AAA ACA |
| NASBA Primer | ACA CGA (SEQ ID NO: 58) |

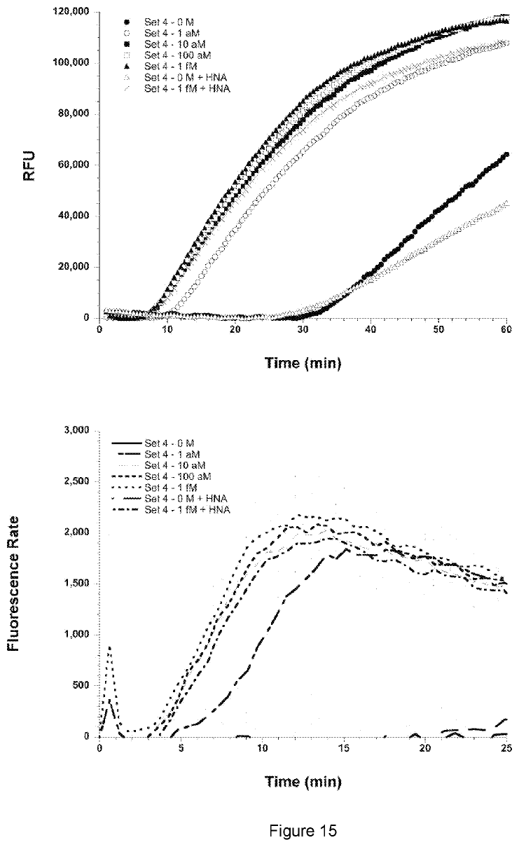

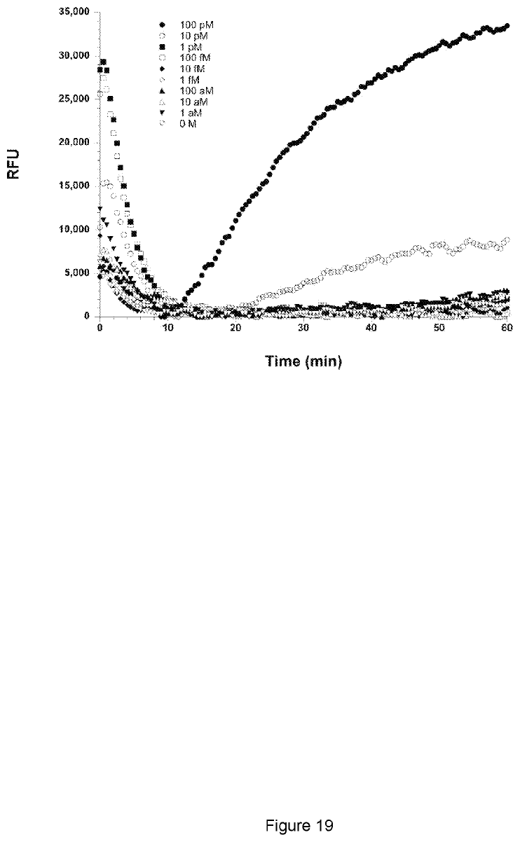

The sensitivity of 1 aM was achieved by performing the dilution series of COVID-19 RNA (1 fM-1 aM) and subjecting it to nested NASBA (FIG. 15A). Addition of 100 ng of exogenous nucleic acid per 20 μl outer reaction did not affect either the positive (grey) or the negative (black) signal (FIG. 15A). LS lyophilized kits were also tested in single step Mango NASBA and demonstrated a sensitivity of 10 pM (FIG. 19).

LS lyophilized kit was compared to the liquid kit results. Serially diluted SARS-CoV-2 RNA (1 fM-1 aM) was subjected to nested NASBA as in FIGS. 15A. FIG. 16 shows that using the lyophilized reagents resulted in a sensitivity of 10 aM and higher.

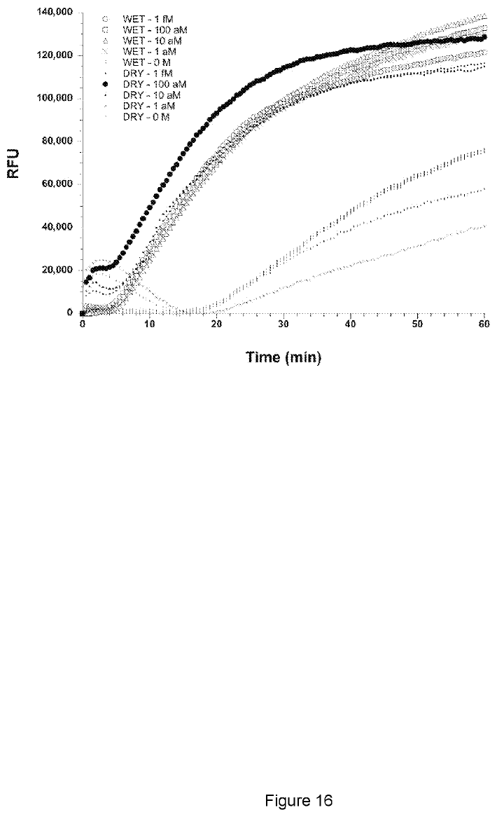

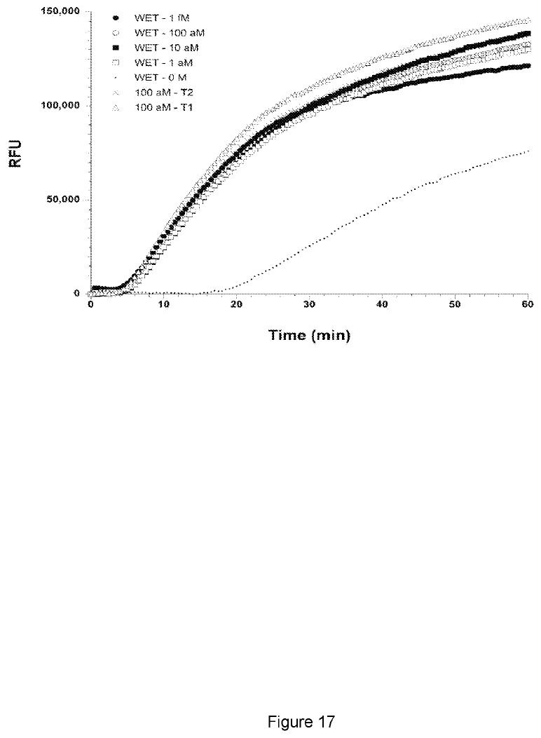

FIG. 17 shows that neither EDTA, nor heating the RNA sample, is required prior to performing the outer nested NASBA reaction.

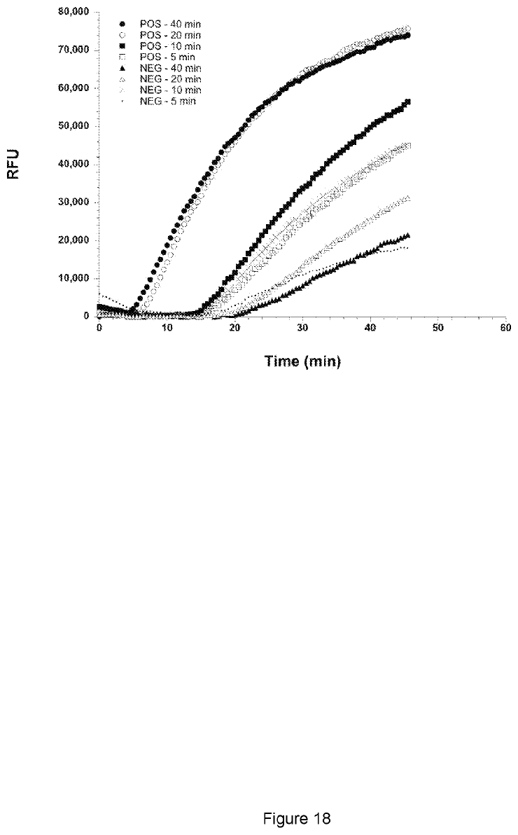

To shorten the overall Mango NASBA reaction time, the outer reaction time was tested using 10 aM SARS-CoV-2 RNA Target 4 (FIG. 18). Outer reaction time of 20 min was demonstrated to maintain the sensitivity and robustness of the 40 min outer incubation time.

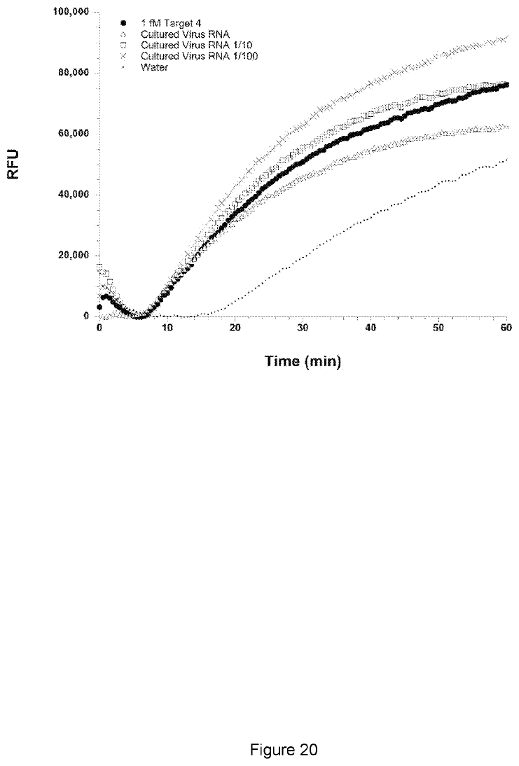

After successful detection of synthetic viral sequences, both liquid and lyophilized LS reagents were tested in Mango NASBA against total RNA extracted from SARS-CoV-2 virus cultured in eukaryotic cells. FIG. 20 (liquid) and FIG. 21 (dry) demonstrated successful detection of cultured virus after a hundred fold dilution of the culture sample (liquid). FIG. 21 also shows that no preheating and a 20 fold dilution from the outer into the inner reaction was fully viable.

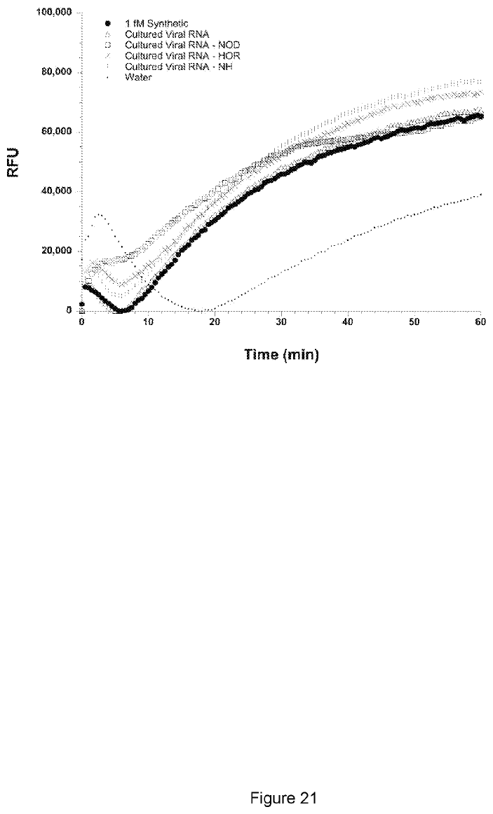

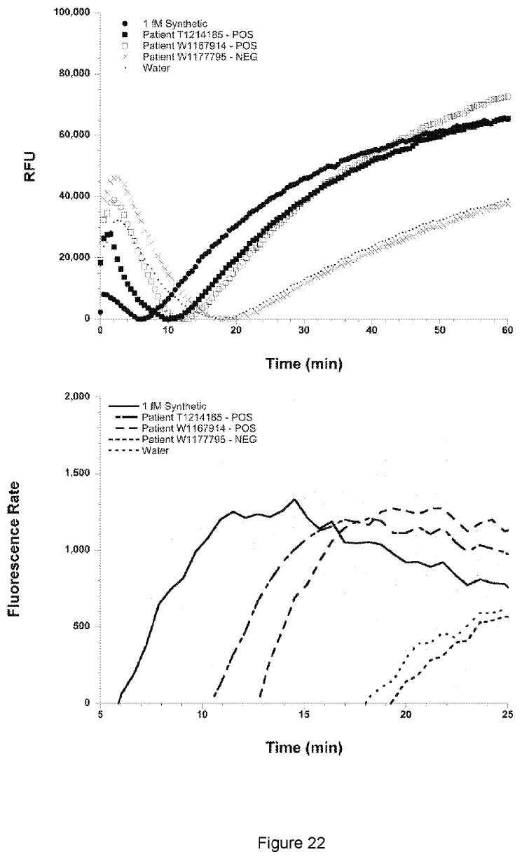

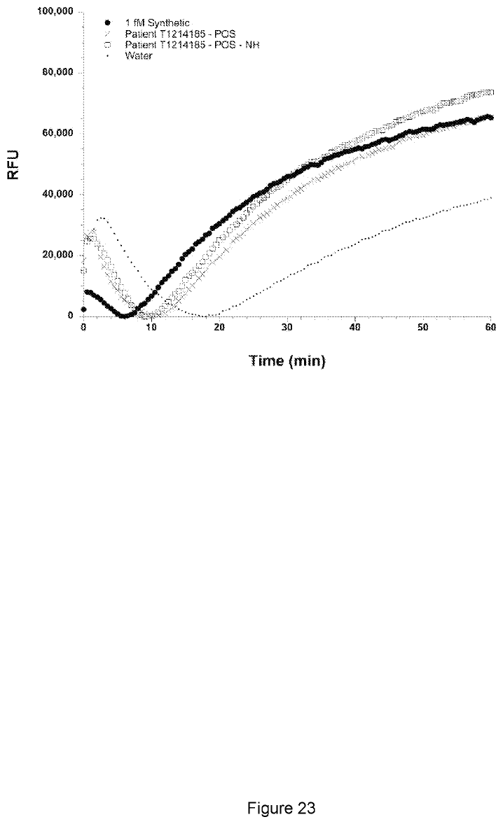

Tracheal aspirates from SARS-CoV-2-infected patients from ICU unit of St Paul's hospital were tested using nested Mango NASBA (FIG. 22), the patients being originally diagnosed by performing Roche RT-PCR test. SARS-CoV-2 RNA was successfully detected in a 20 min outer and 12 min inner NASBA reaction in samples from infected patients (1A and 2A), whereas the curve for the uninfected patient (5A) rose at approximately the same time as the negative control water sample. As with synthetic SARS-CoV-2, heating of the viral RNA sample prior to nested Mango NASBA was not required (FIG. 23).

The commercial lyophilized reagents were slightly turbid at the start of the incubation. This turbidity did not however interfere with analysis and, by plotting the slopes of the data as in FIG. 15B and FIG. 22B, emergence times for each sample could be monitored.

### REFERENCES

1. Niemz, A., Ferguson, T. M. & Boyle, D. S. Point-of-care nucleic acid testing for infectious diseases. Trends in Biotechnology 29, 240-250 (2011).
2. Wang, S. et al. Advances in addressing technical challenges of point-of-care diagnostics in resource-limited settings. Expert Review of Molecular Diagnostics 16, 449-459 (2016).
3. Methods for Rapid Detection of Foodborne Pathogens: An Overview—SciAlert Responsive Version. doi:10.3923/ajft.2011.87.102
4. Law, J. W.-F., Ab Mutalib, N.-S., Chan, K.-G. & Lee, L.-H. Rapid methods for the detection of foodborne bacterial pathogens: principles, applications, advantages and limitations. Frontiers in Microbiology 5, (2015).
5. Zhao, X., Lin, C.-W., Wang, J. & Oh, D. H. Advances in rapid detection methods for foodborne pathogens. J. Microbiol. Biotechnol. 24, 297-312 (2014).
6. Craw, P. & Balachandran, W. Isothermal nucleic acid amplification technologies for point-of-care diagnostics: a critical review. Lab Chip 12, 2469-2486 (2012).
7. Nguyen, T. T., Van Giau, V. & Vo, T. K. Multiplex PCR for simultaneous identification of E. coli O157:H7, Salmonella spp. and L. monocytogenes in food. 3 Biotech 6, (2016).
8. Silva, D. S. P. et al. Multiplex PCR for the simultaneous detection of Salmonella spp. and Salmonella Enteritidis in food. International Journal of Food Science & Technology 46, 1502-1507 (2011).
9. Hongbao, M. Development Application of Polymerase Chain Reaction (PCR). 47 (2005).
10. Velusamy, V., Arshak, K., Korostynska, O., Oliwa, K. & Adley, C. An overview of foodborne pathogen detection: In the perspective of biosensors. Biotechnology Advances 28, 232-254 (2010).
11. Lazcka, O., Campo, F. J. D. & Munoz, F. X. Pathogen detection: A perspective of traditional methods and biosensors. Biosensors and Bioelectronics 22, 1205-1217 (2007).
12. Navarro, E., Serrano-Heras, G., Castaño, M. J. & Solera, J. Real-time PCR detection chemistry. Clinica Chimica Acta 439, 231-250 (2015).
13. Nolan, T., Hands, R. E. & Bustin, S. A. Quantification of mRNA using real-time RT-PCR. Nature Protocols 1, 1559-1582 (2006).
14. Zhao, Y., Chen, F., Li, Q., Wang, L. & Fan, C. Isothermal Amplification of Nucleic Acids. Chem. Rev. 115, 12491-12545 (2015).
15. Lobo-Castañón, M. J. Isothermal nucleic acid amplification in bioanalysis. Analytical and Bioanalytical Chemistry 408, 8581-8582 (2016).
16. Compton, J. Nucleic acid sequence-based amplification. Nature (1991). doi:10.1038/350091 a0
17. Notomi, T. et al. Loop-mediated isothermal amplification of DNA. Nucleic Acids Res 28, e63 (2000).
18. Piepenburg, O., Williams, C. H., Stemple, D. L. & Armes, N. A. DNA Detection Using Recombination Proteins. PLoS Biol 4, (2006).
19. Vincent, M., Xu, Y. & Kong, H. Helicase-dependent isothermal DNA amplification. EMBO Rep 5, 795-800 (2004).
20. Song, J. et al. Two-Stage Isothermal Enzymatic Amplification for Concurrent Multiplex Molecular Detection. Clin Chem 63, 714-722 (2017).
21. Gootenberg, J. S. et al. Nucleic acid detection with CRISPR-Cas13a/C2c2. Science 356, 438-442 (2017).
22. Gootenberg, J. S. et al. Multiplexed and portable nucleic acid detection platform with Cas13, Cas12a, and Csm6. Science 360, 439-444 (2018).
23. Chen, J. S. et al. CRISPR-Cas12a target binding unleashes indiscriminate single-stranded DNase activity. Science (New York, N.Y.) 360, 436-439 (2018).
24. Zhuo, Z. et al. Recent Advances in SELEX Technology and Aptamer Applications in Biomedicine. International Journal of Molecular Sciences 18, 2142 (2017).
25. Dolgosheina, E. V. & Unrau, P. J. Fluorophore-binding RNA aptamers and their applications. Wiley Interdisciplinary Reviews: RNA 7, 843-851 (2016).
26. Bertrand, E. et al. Localization of ASH1 mRNA Particles in Living Yeast. Molecular Cell 2, 437-445 (1998).
27. Mannack, L. V. J. C., Eising, S. & Rentmeister, A. Current techniques for visualizing RNA in cells. F1000Res 5, (2016).
28. Tutucci, E. et al. An improved MS2 system for accurate reporting of the mRNA life cycle. Nat Methods 15, 81-89 (2018).
29. Dolgosheina, E. V. et al. RNA Mango Aptamer-Fluorophore: A Bright, High-Affinity Complex for RNA Labeling and Tracking. ACS Chemical Biology 9, 2412-2420 (2014).
30. Autour, A. et al. Fluorogenic RNA Mango aptamers for imaging small non-coding RNAs in mammalian cells. Nature Communications 9, 656 (2018).
31. Dolgosheina, E. V. & Unrau, P. J. Fluorophore-binding RNA aptamers and their applications. Wiley Interdisciplinary Reviews: RNA 7, 843-851 (2016).
32. Trachman, R. J. et al. Structure and functional reselection of the Mango-III fluorogenic RNA aptamer. Nature Chemical Biology 15, 472 (2019).
33. Autour, A. et al. Fluorogenic RNA Mango aptamers for imaging small non-coding RNAs in mammalian cells. Nature Communications 9, 656 (2018).
34. Fluorescent Visualization of Mango-tagged RNA in Polyacrylamide Gels via a Poststaining Method|Protocol. Available at: https://www.jove.com/video/59112/fluorescent-visualization-mango-tagged-rna-polyacrylamide-gels-via. (Accessed: 8 Apr. 2019)
35. United States Patent Application 20090081670 published Mar. 26, 2009. NICKING AND EXTENSION AMPLIFICATION REACTION FOR THE EXPONENTIAL AMPLIFICATION OF NUCLEIC ACIDS.
36. Walker, G. T. et al. Strand displacement amplification—an isothermal, in vitro DNA amplification technique. Nucleic Acids Res. 20, 1691c Acids Res.n

### Other Embodiments

The present invention has been described with regard to one or more embodiments. However, it will be apparent to persons skilled in the art that a number of variations and modifications can be made without departing from the scope of the invention as defined in the claims. Therefore, although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. By “about” is meant a variance (plus or minus) from a value or range of 5% or less, for example, 0.5%, 1%, 1.5%, 2.0%, 2.5%, 3.0%, 3.5%, 4.0%, 4.5%, 5.0%, etc. In the description, the word “comprising” is used as an open-ended term, substantially equivalent to the phrase “including, but not limited to,” and the word “comprises” has a corresponding meaning. It is to be however understood that, where the words “comprising” or “comprises,” or a variation having the same root, are used herein, variation or modification to “consisting” or “consists,” which excludes any element, step, or ingredient not specified, or to “consisting essentially of” or “consists essentially of,” which limits to the specified materials or recited steps together with those that do not materially affect the basic and novel characteristics of the claimed invention, is also contemplated. Citation of references herein shall not be construed as an admission that such references are prior art to the present invention. All publications are incorporated herein by reference as if each individual publication was specifically and individually indicated to be incorporated by reference herein and as though fully set forth herein. The invention includes all embodiments and variations substantially as hereinbefore described and with reference to the examples and drawings.

1. A nucleic acid molecule, or analog thereof, comprising:
i) a first nucleic acid sequence, capable of hybridizing to at least a portion of a target nucleic acid sequence, or reverse-complement thereof, and further comprising an aptamer-encoding template sequence, wherein the aptamer-encoding template sequence is positioned at the 3′ end of the first nucleic acid sequence; and
ii) a second nucleic acid sequence, capable of hybridizing to at least a portion of a target nucleic acid sequence, or reverse-complement thereof, wherein the 5′ end of the second nucleic acid sequence is covalently attached to the 3′ end of the first nucleic acid sequence, and wherein the 3′ end of the second nucleic acid sequence does not substantially hybridize to the first nucleic acid sequence.

2. The nucleic acid molecule of claim 1 wherein at least the terminal three nucleotides of the 3′ end of the second nucleic acid sequence do not hybridize to the first nucleic acid sequence.

3. The nucleic acid molecule of claim 1 wherein the first nucleic acid sequence is about 20 to about 100 nucleotides in length.

4.-5. (canceled)

6. The nucleic acid molecule of claim 1 wherein the nucleic acid molecule comprises a terminal stem structure and wherein at least the terminal nucleotide of the 5′ end of the second nucleic acid sequence is complementary to at least the terminal nucleotide of the 5′ end of the first nucleic acid to form at least a portion of the terminal stem structure.

7.-9. (canceled)

10. The nucleic acid molecule of claim 1 wherein the second nucleic acid sequence comprises a degenerate sequence.

11. (canceled)

12. The nucleic acid molecule of claim 1 wherein the target nucleic acid sequence is from a virus, a microorganism, a fungus, an animal or a plant, or is a synthetic construct.

13. (canceled)

14. A composition comprising a first nucleic acid molecule in accordance with claim 1.

15. The composition of claim 14 further comprising a second nucleic acid molecule capable of hybridizing to at least a portion of a target nucleic acid sequence, or reverse-complement thereof, and comprising a first RNA polymerase promoter sequence, wherein the first and second nucleic acid molecules form a first primer pair capable of amplifying a first sequence of the target nucleic acid sequence.

16.-19. (canceled)

20. The composition of claim 14 further comprising a third nucleic acid molecule and a fourth nucleic acid molecule,
wherein the third and fourth nucleic acid molecules form a second primer pair capable of amplifying a second sequence of the target nucleic acid molecule,
wherein either the third nucleic acid molecule or the fourth nucleic acid molecule comprises a second RNA polymerase promoter sequence, and
wherein the second primer pair hybridizes to the target nucleic acid molecule at locations external to that of the first primer pair and is capable of amplifying the first sequence and the second sequence.

21.-26. (canceled)

27. The composition of claim 20 further comprising a fifth nucleic acid molecule and a sixth nucleic acid molecule,
wherein the fifth and sixth nucleic acid molecules form a third primer pair capable of amplifying a third sequence of the target nucleic acid molecule,
wherein either the fifth nucleic acid molecule or the sixth nucleic acid molecule comprises a third RNA polymerase promoter sequence,
wherein the third primer pair hybridizes to the target nucleic acid molecule at a location external to that of the first and second primer pairs and is capable of amplifying the first, second and third sequences.

28.-37. (canceled)

38. A kit comprising the nucleic acid molecule in accordance with claim 1, together with instructions for amplification of a target nucleic acid sequence.

39.-40. (canceled)

41. A method of amplifying a target nucleic acid sequence, the method comprising:
i) providing a sample suspected of containing a target nucleic acid molecule;
ii) providing a first nucleic acid molecule in accordance with claim 1;
iii) providing a second nucleic acid molecule capable of hybridizing to at least a portion of the target nucleic acid sequence, or complement thereof, and comprising a first RNA polymerase promoter sequence,
wherein the first and second nucleic acid molecules form a first primer pair capable of amplifying a first sequence of the target nucleic acid sequence; and
iv) performing a first amplification reaction comprising the target nucleic acid molecule and the first primer pair to obtain a first amplification product, wherein the first amplification product comprises the first sequence of the target nucleic acid sequence.

42.-45. (canceled)

46. The method of claim 41, the method further comprising:
v) providing a third nucleic acid molecule and a fourth nucleic acid molecule,
wherein the third and fourth nucleic acid molecules form a second primer pair capable of amplifying a second sequence of the target nucleic acid molecule,
wherein either the third nucleic acid molecule or the fourth nucleic acid molecule comprises a second RNA polymerase promoter sequence,
wherein the second primer pair hybridizes to the target nucleic acid molecule at a location external to that of the first primer pair and is capable of amplifying the first sequence and the second sequence of the target nucleic acid molecule; and
vi) performing a second amplification reaction comprising the first amplification product and the second primer pair to obtain a second amplification product, wherein the second amplification reaction is performed prior to the first amplification reaction and wherein the second amplification product comprises the first sequence and the second sequence of the target nucleic acid molecule.

47.-52. (canceled)

53. The method of claim 41 further comprising detecting the target nucleic acid sequence.

54. The method of claim 41 further comprising quantifying the target nucleic acid sequence.

55. The method of claim 41 wherein the amplification is an isothermal amplification.

56.-60. (canceled)

61. The method of claim 41 wherein the sample is from a virus, a microorganism, a fungus, an animal, a plant or from the environment.

62.-63. (canceled)

64. The method of claim 61 wherein the virus irus is SARS, MERS or SARS-CoV-2.

65. The method of claim 41 wherein the sample is obtained from water, soil, saliva, feces, urine, blood, tracheal aspirate or nasal aspirate.

66. The method of claim 61 wherein the animal is a human.


SEQUENCE LISTING (58 sequences)

SEQ ID NO: 1 - DNA, 247 bp, E. coli
ggacgtctggaagaacgtggttatgaaatccacatttctgacgaggcgctgaaactgctgagcgagaacggttacgatccggtctatggtgcacgtcctctgaaacgtgcaattcagcagcagatcgaaaacccgctggcacagcaaatactgtctggtgaattggttccgggtaaagtgattcgcctggaagttaatgaagaccggattgtcgccgtccagtaaatgataaaacgagcccttcggg

SEQ ID NO: 2 - DNA, 247 bp, P. fluorescens
ggtcgcctggccgagcgtgagcttgacctggagctgagcagcgaggcgttggacaagctgattgcggtcggttacgacccggtgtatggcgcacggccacttaaacgtgcgatccagcgctggatcgaaaacccactggcacagttgatcctgtcgggcagcttcatgccaggcacccgcgtgacggccacggtggaaaacgacgaaatcgtcttccactaagcccagcctgtagggttattagaga

SEQ ID NO: 3 - RNA, 247 nt, E. coli
ggacgucuggaagaacgugguuaugaaauccacauuucugacgaggcgcugaaacugcugagcgagaacgguuacgauccggucuauggugcacguccucugaaacgugcaauucagcagcagaucgaaaacccgcuggcacagcaaauacugucuggugaauugguuccggguaaagugauucgccuggaaguuaaugaagaccggauugucgccguccaguaaaugauaaaacgagcccuucggg

SEQ ID NO: 4 - RNA, 247 nt, P. fluorescens
ggucgccuggccgagcgugagcuugaccuggagcugagcagcgaggcguuggacaagcugauugcggucgguuacgacccgguguauggcgcacggccacuuaaacgugcgauccagcgcuggaucgaaaacccacuggcacaguugauccugucgggcagcuucaugccaggcacccgcgugacggccacgguggaaaacgacgaaaucgucuuccacuaagcccagccuguaggguuauuagaga

SEQ ID NO: 5 - DNA, 45 bp, Artificial Sequence
  Primer
ctttaatacgactcactataggacgtctggaagaacgtggttatg

SEQ ID NO: 6 - DNA, 25 bp, Artificial Sequence
  Primer
cccgaagggctcgttttatcattta

SEQ ID NO: 7 - DNA, 56 bp, Artificial Sequence
  Primer
aattctaatacgactcactatagggagaaggctggacggcgacaatccggtcttca

SEQ ID NO: 8 - DNA, 67 bp, Artificial Sequence
  Primer
ggcacgtacgaatataccacataccaaaccttccttcgtacgtgccaaatccacatttctgacgagg

SEQ ID NO: 9 - DNA, 21 bp, Artificial Sequence
  Primer
aaatccacatttctgacgagg

SEQ ID NO: 10 - DNA, 67 bp, Artificial Sequence
  Primer
ggcacgtacgaatataccacataccaatccttccttcgtacgtgccaaatccacatttctgacgagg

SEQ ID NO: 11 - DNA, 51 bp, Artificial Sequence
  Primer
aattctaatacgactcactatagggaaggaagtctggtgaattggttccgg

SEQ ID NO: 12 - DNA, 66 bp, Artificial Sequence
  Primer
ggcacgtacgaatataccacataccaaaccttccttcgtacgtgccttccaggcgaatcactttac

SEQ ID NO: 13 - DNA, 45 bp, Artificial Sequence
  Primer
ctttaatacgactcactataggtcgcctggccgagcgtgagcttg

SEQ ID NO: 14 - DNA, 25 bp, Artificial Sequence
  Primer
tctctaataaccctacaggctgggc

SEQ ID NO: 15 - DNA, 43 bp, Artificial Sequence
  Primer
ctttaatacgactcactatagggaggctgggcttagtggaaga

SEQ ID NO: 16 - DNA, 20 bp, Artificial Sequence
  Primer
gagcagcgaggcgttggaca

SEQ ID NO: 17 - DNA, 43 bp, Artificial Sequence
  Primer
ctttaatacgactcactatagggcgaaaacccactggcacagt

SEQ ID NO: 18 - DNA, 66 bp, Artificial Sequence
  Primer
ggcacgtacgaatataccacataccaaaccttccttcgtacgtgcccgcgggtgcctggcatgaag

SEQ ID NO: 19 - DNA, 100 bp, Artificial Sequence
  Linear RCA template
aagttttcagctgcttgccctatagtgagtcgtattaggcacgtacgaatataccacataccaatccttccttcgtacgtgcccggaaaagtttgaagag

SEQ ID NO: 20 - DNA, 65 bp, Artificial Sequence
  Target RCA splint
aaaagcggaaaagtttgaagagaagttttcagctgcttgcgcttatcctatagtgagtcgtatta

SEQ ID NO: 21 - DNA, 22 bp, Artificial Sequence
  T7 promoter top strand sequence
ctttaatacgactcactatagg

SEQ ID NO: 22 - DNA, 50 bp, Artificial Sequence
  Primer
ctttaatacgactcactataggggtgcaaggttacaagagtgtgaatatc

SEQ ID NO: 23 - DNA, 30 bp, Artificial Sequence
  Primer
acacaaactcttaaagaatgtatagggtca

SEQ ID NO: 24 - DNA, 20 bp, Artificial Sequence
  Primer
gtagacatttgtgcgaacag

SEQ ID NO: 25 - DNA, 55 bp, Artificial Sequence
  Primer
ctttaatacgactcactatagggtgtatacattaaaaatgcagacattgtggaag

SEQ ID NO: 26 - DNA, 29 bp, Artificial Sequence
  Primer
cagttccaagaatttcttgcttctcatta

SEQ ID NO: 27 - DNA, 20 bp, Artificial Sequence
  Primer
agcatttctcgcaaattcca

SEQ ID NO: 28 - DNA, 50 bp, Artificial Sequence
  Primer
ctttaatacgactcactatagggatctttataagctcatgggacacttcg

SEQ ID NO: 29 - DNA, 37 bp, Artificial Sequence
  Primer
ttaataggcgtgtgcttagaatatattttaaaataac

SEQ ID NO: 30 - DNA, 20 bp, Artificial Sequence
  Primer
accctgagggagatcacgca

SEQ ID NO: 31 - DNA, 47 bp, Artificial Sequence
  Primer
ctttaatacgactcactatagggtgccactagtctctagtcagtgtg

SEQ ID NO: 32 - DNA, 26 bp, Artificial Sequence
  Primer
aacttcaccaaaagggcacaagtttg

SEQ ID NO: 33 - DNA, 20 bp, Artificial Sequence
  Primer
cagatgcaaatctggtggcg

SEQ ID NO: 34 - DNA, 59 bp, Artificial Sequence
  Primer
ctttaatacgactcactatagggaagaaagacagaatgattgaactttcattaattgac

SEQ ID NO: 35 - DNA, 25 bp, Artificial Sequence
  Primer
gattgcagcattgttagcaggattg

SEQ ID NO: 36 - DNA, 20 bp, Artificial Sequence
  Primer
ttgttccttgaggaagttgt

SEQ ID NO: 37 - DNA, 43 bp, Artificial Sequence
  Primer
ctttaatacgactcactatagggttccatctctaattgaggtt

SEQ ID NO: 38 - DNA, 20 bp, Artificial Sequence
  Primer
tagtcaacaaactgttggtc

SEQ ID NO: 39 - DNA, 43 bp, Artificial Sequence
  Primer
ctttaatacgactcactataggggcagtgaggacaatcagaca

SEQ ID NO: 40 - DNA, 66 bp, Artificial Sequence
  Primer
ggcacgtacgaatataccacataccaaaccttccttcgtacgtgccacaattgtttgaatagtagt

SEQ ID NO: 41 - DNA, 43 bp, Artificial Sequence
  Primer
ctttaatacgactcactatagggctttcagttataaatggctt

SEQ ID NO: 42 - DNA, 20 bp, Artificial Sequence
  Primer
agctttttggaaatgaagag

SEQ ID NO: 43 - DNA, 43 bp, Artificial Sequence
  Primer
ctttaatacgactcactataggggcaagttgaacaaaagatcg

SEQ ID NO: 44 - DNA, 66 bp, Artificial Sequence
  Primer
ggcacgtacgaatataccacataccaaaccttccttcgtacgtgccttcctctttaggaatctcag

SEQ ID NO: 45 - DNA, 43 bp, Artificial Sequence
  Primer
ctttaatacgactcactatagggggaaaagaaaggtaagaaca

SEQ ID NO: 46 - DNA, 20 bp, Artificial Sequence
  Primer
taccccctgcatacactaat

SEQ ID NO: 47 - DNA, 43 bp, Artificial Sequence
  Primer
ctttaatacgactcactatagggtaccctgacaaagttttcag

SEQ ID NO: 48 - DNA, 66 bp, Artificial Sequence
  Primer
ggcacgtacgaatataccacataccaaaccttccttcgtacgtgccttgaatgtaaaactgaggat

SEQ ID NO: 49 - DNA, 43 bp, Artificial Sequence
  Primer
ctttaatacgactcactatagggtaagaacaagtcctgagttg

SEQ ID NO: 50 - DNA, 66 bp, Artificial Sequence
  Primer
ggcacgtacgaatataccacataccaaaccttccttcgtacgtgcccagatcctcagttttacatt

SEQ ID NO: 51 - DNA, 43 bp, Artificial Sequence
  Primer
ctttaatacgactcactatagggttccctaagatttttgaaat

SEQ ID NO: 52 - DNA, 20 bp, Artificial Sequence
  Primer
agtttattctagtgcgaata

SEQ ID NO: 53 - DNA, 43 bp, Artificial Sequence
  Primer
ctttaatacgactcactatagggtttgaatatgtctctcagcc

SEQ ID NO: 54 - DNA, 66 bp, Artificial Sequence
  Primer
ggcacgtacgaatataccacataccaaaccttccttcgtacgtgcctccttcaaggtccataagaa

SEQ ID NO: 55 - DNA, 43 bp, Artificial Sequence
  Primer
ctttaatacgactcactatagggttttagtttgttcgtttaga

SEQ ID NO: 56 - DNA, 20 bp, Artificial Sequence
  Primer
gttgttcgttctatgaagac

SEQ ID NO: 57 - DNA, 43 bp, Artificial Sequence
  Primer
ctttaatacgactcactatagggtttttagagtatcatgacgt

SEQ ID NO: 58 - DNA, 66 bp, Artificial Sequence
  Primer
ggcacgtacgaatataccacataccaaaccttccttcgtacgtgcctgaaatctaaaacaacacga